(19) United States
(12) Patent Application Publication
(10) Pub. No.: US 2024/0371529 A1
(43) Pub. Date: Nov. 7, 2024

(54) AUTOMATICALLY DETECTING ERRORS IN COMPUTER-BASED ANALYTIC METHOD ROUTING OBJECTS

(71) Applicant: Providence St. Joseph Health, Renton, WA (US)

(72) Inventors: Eve Cunningham, Tacoma, WA (US); John Tapsell, Irvine, CA (US); Adrian Yanes, Santa Barbara, CA (US); Robert Fearn, Irvine, CA (US); Erin Longley, Tacoma, WA (US)

(21) Appl. No.: 18/311,757

(22) Filed: May 3, 2023

(51) Int. Cl. G16H 70/00

(52) U.S. Cl. CPC G16H 70/00

(57) ABSTRACT

A method for diagnosing errors in an analytic method definition is described. The facility accesses the definition, which specifies a set of pages, one or more of which include at least one question. Each question has two or more possible answers. One or more pages each include a swimlane control having a plurality of lanes, each lane identifying a combination of possible answers to two or more of the questions included among the pages and specifying routing in an iteration of the analytic method in which that combination of answers has been given. For each of the swimlane controls, among the included questions any of whose answers have been included in the combination of answers identified by a lane of the swimlane, the facility determines any combinations of possible answers not identified by any lane of the swimlane.

### CROSS-REFERENCE TO RELATED APPLICATION

This Application is related to U.S. application Ser. No. 17/955,296, filed Sep. 28, 2022, and entitled “CONSTRUCTING AND APPLYING COMPUTER-BASED ANALYTIC METHODS REPRESENTING MEDICAL KNOWLEDGE, and U.S. application Ser. No. 17/955,298, filed Sep. 28, 2022, and entitled “CONSTRUCTING AND APPLYING COMPUTER-BASED ANALYTIC METHODS REPRESENTING MEDICAL KNOWLEDGE,” each of which is hereby incorporated by reference in its entirety.

In cases where the present application conflicts with a document incorporated by reference, the present application controls.

### BACKGROUND

Medical professionals who treat patients (“caregivers”) rely on medical knowledge. For many caregivers, this medical knowledge can extend to an extensive variety of different kinds of information: normal and anomalous body function; condition diagnosis, including relevant forms of physical examination and testing; referral to specialists; condition treatment options and attendant benefits and risks, including by prescribing medicines or placebos, performing surgery, undergoing physical therapy; surgical and anesthesiological techniques; epidemiological updates; techniques for interacting with patients and cooperating caregivers; etc.

Medical knowledge is obtained by caregivers from a variety of sources, such as: medical school classes and textbooks; clinical rotations, internships, residencies, and fellowships; treating patients; ongoing medical education seminars and conferences; informal discussions with other caregivers; reading journal articles; reviewing pharmaceutical data sheets; consulting treatment or referral guides and other information published by or within a caregiver's organization; etc.

### BRIEF DESCRIPTION OF THE DRAWINGS

FIG. 1 is a block diagram showing some of the components typically incorporated in at least some of the computer systems and other devices on which the facility operates.

FIG. 2 is a data flow diagram providing a high-level view of the operation of the facility in some embodiments.

FIG. 3 is a flow diagram showing a process performed by the facility in some embodiments to build an analytic method.

FIG. 4 is a display diagram showing a sample display presented by the facility in some embodiments to begin the creation of a first page of a new analytic method using the creator user interface.

FIG. 5 is a display diagram showing a sample display presented by the facility in some embodiments to add textual content to a page.

FIG. 6 is a display diagram showing a sample display presented by the facility in some embodiments to enter information about a question to be posed by an analytic method page.

FIG. 7 is a display diagram showing a sample display presented by the facility in some embodiments to define possible answers to a question.

FIG. 8 is a display diagram showing a sample display presented by the facility in some embodiments to define an additional question for the sample analytic method's starting page.

FIG. 9 is a display diagram showing a sample display presented by the facility in some embodiments to enter possible answers to the second question.

FIG. 10 is a flow diagram showing a process performed by the facility in some embodiments to include a swimlane in an analytic method to specify routing based upon the answers to multiple questions.

FIG. 11 is a display diagram showing a sample display presented by the facility in some embodiments to begin definition of a swimlane.

FIG. 12 is a display diagram showing a sample display presented by the facility in some embodiments to define a first column of a swimlane.

FIG. 13 is a display diagram showing a sample display presented by the facility in some embodiments to define additional columns in a swimlane.

FIG. 14 is a display diagram showing a sample display presented by the facility in some embodiments to specify the routing target for a particular swimlane column.

FIG. 15 is a display diagram showing a sample display presented by the facility in some embodiments to enter content to display if a consumer submits the combination of answers to which the leftmost swimlane column corresponds.

FIG. 16 is a display diagram showing a sample display presented by the facility in some embodiments to display content previously defined for a swimlane column.

FIG. 17 is a display diagram showing a sample display presented by the facility in some embodiments to specify a routing target for the fourth through sixth columns of the swimlane.

FIG. 18 is a display diagram showing a sample display presented by the facility in some embodiments to create a new page that was specified as the target of the fourth through sixth columns of the swimlane.

FIG. 19 is a display diagram showing a sample display presented by the facility in some embodiments to enter content for the no signs of inflammation page.

FIG. 20 is a display diagram showing a sample display presented by the facility in some embodiments to add a question to the no signs of inflammation page.

FIG. 21 is a flow diagram showing a process performed by the facility in some embodiments in order to permit navigation of the analytic method via the graph diagram.

FIG. 22 is display diagram showing a sample display presented by the facility in some embodiments to begin navigating via the graph diagram visualization.

FIG. 23 is a display diagram showing a sample display presented by the facility to reflect navigation to the second page of the analytic method.

FIG. 24 is a display diagram showing sample contents of a display presented by the facility in some embodiments to navigate to the third page of the analytic method.

FIG. 25 is a flow diagram showing a process performed by the facility in some embodiments to apply an analytic method on behalf of a consumer user, such as one using the facility with respect to a particular patient.

FIG. 26 is a display diagram showing a display presented by the facility in some embodiments to begin application of an analytic method.

FIG. 27 is a display diagram showing a sample display presented by the facility in some embodiments to expand an information icon.

FIG. 28 is a display showing a sample display presented by the facility in some embodiments to display a second page of the sample analytic method in response to the consumer's answering of the questions of the first page.

FIG. 29 is a display diagram showing a sample display presented by the facility in some embodiments to make visible the remainder of the second page of the sample analytic method.

FIG. 30 is a display diagram showing a sample display presented by the facility in some embodiments to show the routing flow taken by the facility in the sample analytic method based upon the consumer's interactions in the third page.

FIG. 31 is a flow diagram showing a process performed by the facility in some embodiments to perform a diagnostic assessment of an analytic method.

FIG. 32 is a flow diagram showing a process performed by the facility in some embodiments to handle a call to the visit_page function.

FIG. 33 is a display diagram showing a first display presented by the facility in some embodiments to show the results of the facility's diagnostic assessment for a sample analytic method.

FIG. 34 is a display diagram showing a second display presented by the facility in some embodiments to show the results of the facility's diagnostic assessment for a sample analytic method.

### DETAILED DESCRIPTION

The inventors have recognized significant disadvantages in the conventional ways that medical knowledge is created and disseminated to medical caregivers such as physicians and applied by them.

In particular, a staggering volume of medical knowledge, in widely-varying subject matter areas, can be beneficially applied to the treatment of many caregivers' patients. This beneficial medical knowledge is often both within the caregiver's specialty area, and in adjacent or related specialty areas. Caregivers are unable to reliably retain all of the medical knowledge they have encountered.

Also, whether consumed in advance of needing it or directly in response to needing it, medical knowledge is spread over a wide variety of sources and modalities that makes it difficult to locate pieces of medical knowledge relevant to a patient situation. Also, in many cases it is difficult to assess the accuracy of a particular piece of medical knowledge, or select the most useful of multiple sources for particular medical knowledge. There is often also a trade-off between reliability and latency of sources of medical knowledge, in light of the significant length of the authoring, review, and publication cycle of medical journal articles. Additional factors can also make medical knowledge obsolete as soon as it is made available, or shortly thereafter.

It is common for sources of medical knowledge to be difficult to apply to a caregiver's needs. For example, certain sources may be focused on theory, to the exclusion of practice, or practical aspects may be difficult to locate among content that is less practical. Some sources that seek to provide practical guidance achieve limited success based on using ill-suited language, formatting, or organization, for example.

Because of the high level of dynamism of many medical fields, caregivers often face the challenge of medical knowledge “changing out from under them”: both caregiver memories and static sources of medical knowledge can become outdated as new discoveries are made, new technologies emerge and mature, new techniques and best practices are developed, etc.

In response to recognizing these disadvantages, the inventors have conceived and reduced to practice a software and/or hardware facility for constructing and applying computer-based analytic methods representing medical knowledge (“the facility”). These analytic methods can represent a wide variety of different kinds of medical knowledge, including, for example, disorder diagnoses, referral recommendations, medical procedures or protocols, selection among condition treatment options, etc.

In some embodiments, the facility provides a creator user interface for creating and revising analytic methods, as well as one or more other user interfaces for consuming and/or visualizing analytic methods.

In some embodiments, a “creator” user—such as a domain expert, or another qualified person who is transforming a conventional source of medical knowledge into an analytic method—interacts with controls presented by the facility in order to create an analytic method for use by consumer users, such as caregivers who wish to rely on the medical knowledge embodied in the analytic method. In particular, the creator creates a set of “pages” that make up the analytic method, which can correspond to individual “blocks of knowledge.” In each page, the creator can specify questions, their possible answers, and whether it is valid for more than one of the possible answers to be selected at the same time by a consumer. For a particular question, the creator can specify routing that is to occur in response to each of the question's possible answers being given. This routing can be to destinations of various types, including (1) certain text or other content that the creator specifies within the context of the page; (2) another page generated by the facility, either part of the current analytic method or a different one, either one that already exists or that the creator will create; (3) a document created outside the facility; or (4) an action, such as against an electronic medical record (“EMR”) software system with respect to the patient who is the subject of a consumer's use of the consumer user interface. In some embodiments, the facility permits the creator to add small supplemental information icons to particular portions of a page, which can be expanded to display supplemental informational content about that portion of the page in the consumer user interface, such as by hovering over or touching or clicking on the information icon.

In some embodiments, the facility provides a “swimlane” control in which the creator can use a visual grid scheme to easily specify routing for different combinations of answers to a group of multiple questions. For example, where a page in analytic method relating to joint pain contains a first question asking how many joints are affected—whose answers include one, two, and more than two—and a second question asking whether those joints are swollen—whose answer include yes and no—the creator can use the swimlane to specify one routing target for the combination of the “one” answer to the first question and the “yes” answer to the second question; another routing target for the combination of the “one” answer to the first question and the “no” answer to the second question; etc. In some embodiments, the facility enables the creator to group multiple answer combinations together with the same routing target.

In some embodiments, as the creator uses the creator user interface to construct the analytic method, the facility concurrently displays and updates a graph diagram visualization of the analytic method in which each page is shown as a node, and each routing path to another page is shown as an edge between nodes. In some embodiments, the creator can select one of the nodes of the graph diagram to jump to the corresponding page in the creator user interface. In some embodiments, the creator can interact with one of the nodes of the graph diagram to delete or otherwise alter the corresponding page of the analytic method.

In some embodiments, as part of the creator interface, the creator can switch on an interview mode in order to preview the experience that consumers will have in using the analytic method. If the creator identifies an issue with a page in the interview mode, the creator can switch the interview mode off in order to alter the definition of the page in the creator user interface.

In some embodiments, throughout the creation of the analytic method, the facility continuously updates an internal representation of the analytic method that is stored by the facility to maintain the persistence of the analytic method, and is a basis for rendering all of the ways supported by the facility to visualize and interact with the analytic method, including the creator user interface, the graph diagram visualization, and consumer interview user interfaces. In various embodiments, this internal representation is stored locally in a browser and/or on a server.

By using the facility to represent medical knowledge as an analytic method in the ways described above, the facility renders that medical knowledge “computable”—i.e., usable in the facility's consumer user interface to directly and systematically apply to a patient.

In some embodiments, the facility provides one or more consumer user interfaces for applying analytic methods created using the facility, such as interview user interfaces in which an analytic method's questions are presented to a consumer; answers to the questions are received from the consumer; and content, additional questions, and/or actions routed to according to the received answers are presented to the consumer. In various embodiments, these interview user interfaces conduct their interviews in a variety of modalities. These can include a touch- or click-based visual user interface in which answers to questions are displayed as buttons that the consumer can select by touching them or clicking on them; a text-based visual user interface in which consumers type answers to their questions, or shorter text strings representing those answers; a voice-based audio user interface in which the facility speaks questions and their answers using speech synthesis, and consumers speak their answers; or a combination of these, e.g., a consumer speaks in response to seeing questions and their possible answers displayed in the touch- or click-based visual user interface.

In some embodiments, the consumer user interface enables a consumer to select a particular analytic method from an inventory of analytic methods contained in a repository; enables a consumer to search for or otherwise discover analytic methods of this inventory appropriate to a particular patient scenario; etc.

In some embodiments, the consumer user interface concurrently displays the graph diagram visualization of the selected analytic method, enabling the consumer to follow their progress through the analytic method, skip forward or backward among the pages of the analytic method; switch to a different route among the pages of the analytic method, jump to a different analytic method, etc.

The inventors have further noted certain errors that creators sometimes make in creating an analytic method. These include (1) creating pages in the analytic method that are unreachable from the analytic method's root page; (2) creating loops among the connections between pages in the analytic method; (3) when creating a swimlane control for specifying routing for different combinations of answers to a group of multiple questions, omitting lanes that provide routing for certain possible combinations of answers; (4) creating unreachable swimlanes; (5) linking to external documents that don't exist; (6) linking to a specific page that doesn't exist in an external document; and (7) linking to an external document in a different audience (e.g. an adult guide accidentally linking to a pediatric-only guide, without explicitly marking it as such).

In response to this recognition, in some embodiments, the facility automatically performs diagnostic assessments of created analytic methods, such as continuously during their creation, or at certain junctures, such as when each page is saved, or when the analytic method's creation is completed. In various embodiments, these diagnostic assessments include (1) whether any page of the analytic method is unreachable from the root page; (2) whether the connections between the pages of the analytic method form any loop; and/or (3) in each swimlane control, whether lanes that provide routing for any possible combinations of answers have been omitted.

By performing some or all of the ways described above, the facility makes it straightforward to embody medical knowledge in a computer-based analytic method, either medical knowledge originated by the creator of the analytic method or medical knowledge originated by one or more third-party sources. This analytic method can be quickly and easily employed by vast numbers of caregivers as part of their treatment of patients, or their ongoing medical studies. The analytic method can later be updated or otherwise improved, to the benefit of this large number of caregivers and their patients. In addition, in some embodiments the facility helps creators to identify and resolve errors in the analytic methods they create.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by concentrating easily-applied medical knowledge in a central repository of analytic methods, the facility reduces the network and processing resources that would otherwise have been used to search for, retrieve, evaluate, and seek to apply conventional sources of medical knowledge. Also, by using a compact internal representation of analytic methods, the facility reduces the amount of storage that would otherwise have been used to store less data-efficient conventional sources of medical knowledge. Further, by automatically assisting creators to identify and correct errors in creating analytic methods, the facility reduces the processing load on computing systems by (1) preventing the unsuccessful use in production of analytic methods that have these errors, which would have to later be repeated once the errors are found and corrected, and (2) obviating the user interface load imposed by creators manually searching for and resolving these errors.

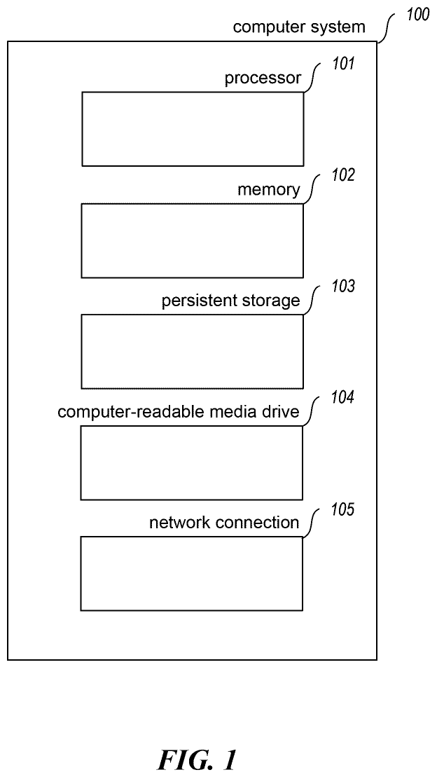

FIG. 1 is a block diagram showing some of the components typically incorporated in at least some of the computer systems and other devices on which the facility operates. In various embodiments, these computer systems and other devices 100 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 101 for executing computer programs and/or training or applying machine learning models, such as a CPU, GPU, TPU, NNP, FPGA, or ASIC; a computer memory 102 for storing programs and data while they are being used, including the facility and associated data, an operating system including a kernel, and device drivers; a persistent storage device 103, such as a hard drive or flash drive for persistently storing programs and data; a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

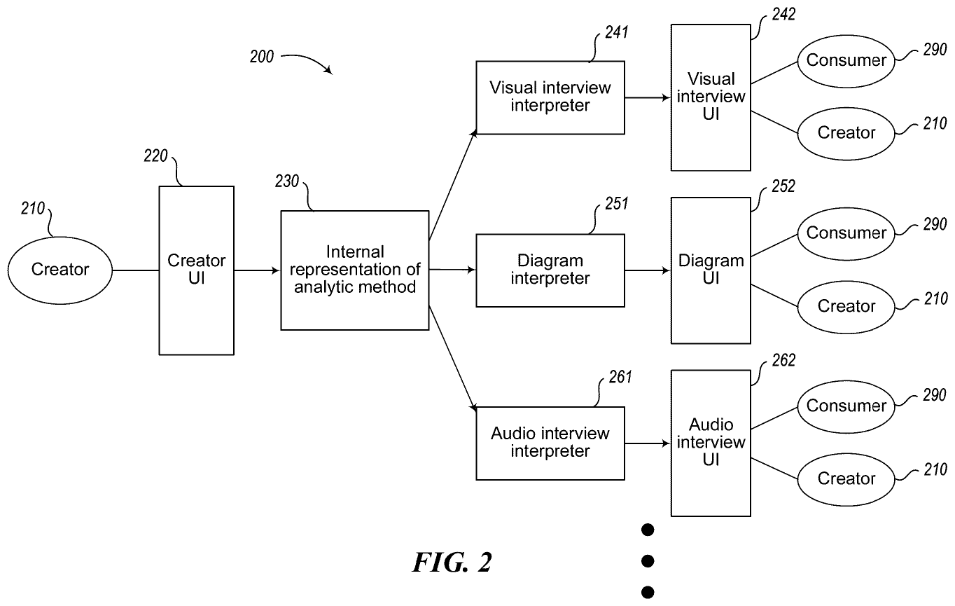

FIG. 2 is a data flow diagram providing a high-level view of the operation of the facility in some embodiments. A creator user 210 interacts with a creator UI 220 provided by the facility in order to create or revise an internal representation of an analytic method 230. The facility stores this internal representation persistently, such as in a repository of analytic methods. The facility provides a variety of interpreters to transform an analytic method's internal representation into a visualization or user interface for the analytic method. For example, a visual interview interpreter 241 interprets the internal representation to generate a visual interview user interface for the analytic method that can be used by any number of consumers 290, as well as the creator to preview the consumer experience of the analytic method. Similarly, a diagram interpreter 251 interprets the internal representation to generate a graph diagram user interface 252 for the analytic method in which the pages of the analytic method are represented as graph nodes; this diagram user interface can similarly be used by consumers or the creator. Further, the facility provides an audio interview interpreter 261 to transform the internal representation of the analytic method into an audio interview user interface for the analytic method, which can be used both by consumers and the creator. In various embodiments, the facility provides additional interpreters as basis for making additional user interfaces available for the analytic method.

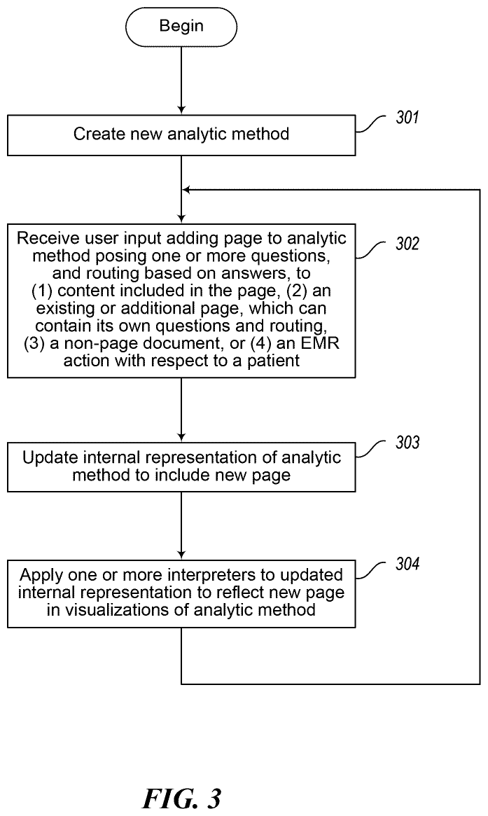

FIG. 3 is a flow diagram showing a process performed by the facility in some embodiments to build an analytic method. In act 301, the facility creates a new analytic method. In some embodiments, act 301 involves receiving metadata for the analytic method from its creator, such as a name, subject matter area, keywords, creator name, creator organization, creator title, creator qualifications or designations, groups of consumer users that can or should use it, etc. The facility initiates storage for the analytic method, and stores the collected metadata for the analytic method. In act 302, the facility receives user input adding a page to the analytic method. This page poses one or more questions, and performs routing based on the answers to those questions. In various embodiments, the routing can be to any of text or other content that is stored as part of the page; a different page-one that already exists or can be subsequently created by the creator-which can contain its own questions and routing; an external document that is not part of an analytic method; or an action performed by an EMR with respect to a patient on whose behalf the analytic method is being applied. In some embodiments, certain pages added to the analytic method by the facility contain no questions; in some such embodiments, routing from these pages is performed unconditionally to a single target, while in others, routing is performed based upon factors other than answers to questions in the page, such as answers to questions in previous pages; in other cases, such a page may be a “leaf” from which no routing to another page occurs.

In act 303, the facility updates the internal representation of the analytic method to include the new page added in act 302. In act 304, the facility applies one or more interpreters to the internal representation updated in act 303 to reflect the new page in visualizations of the analytic method that are presently being used. After act 304, the facility continues in act 302 to receive user input needed to add the next page to the analytic method. Though not shown, in some embodiments, the facility performs acts 302-303 to delete an existing page from the analytic method, revise the content of an existing page, alter routing among pages, split a page into multiple pages, etc.

Those skilled in the art will appreciate that the acts shown in FIG. 3 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

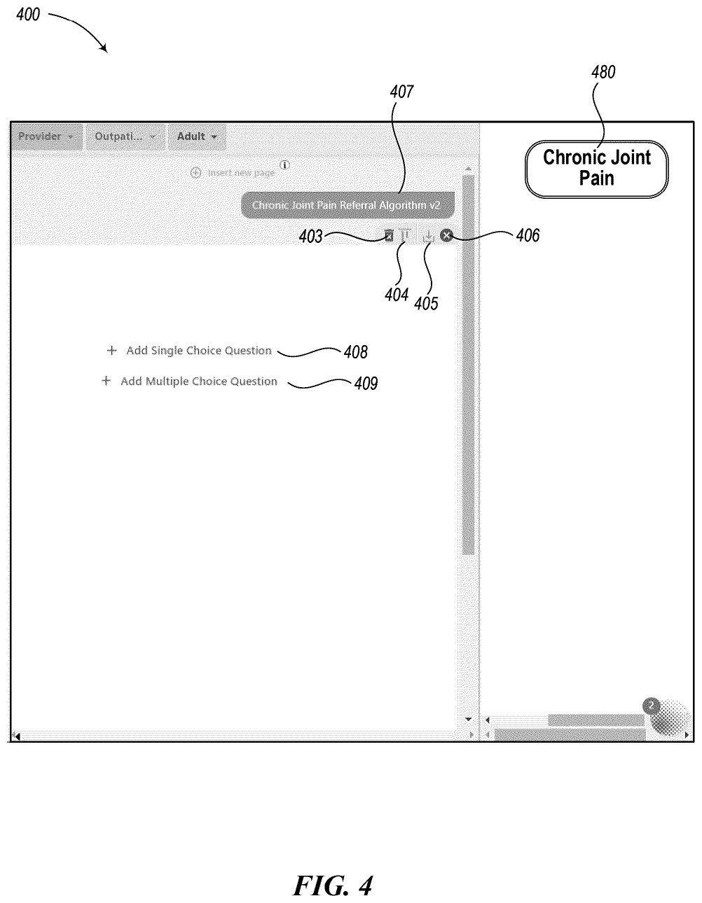

FIG. 4 is a display diagram showing a sample display presented by the facility in some embodiments to begin the creation of a first page of a new analytic method using the creator user interface. The display 400 includes an indication 407 of a name specified for the analytic method by the creator, as well as controls 408 and 409 for adding questions to the analytic method's first page. For example, the user can click control 408 in order to add a single choice question to the first page.

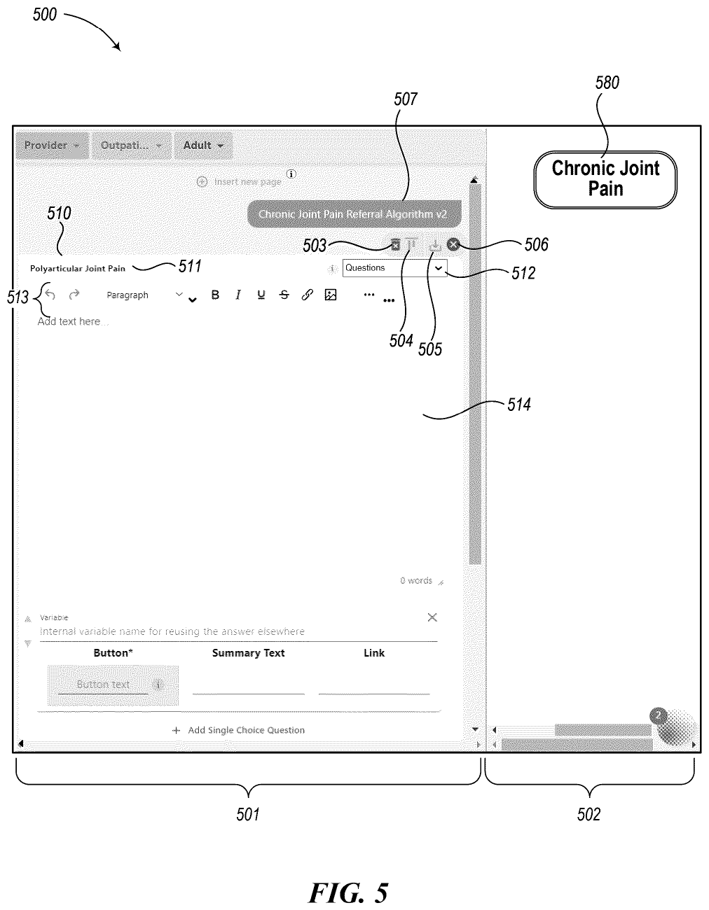

FIG. 5 is a display diagram showing a sample display presented by the facility in some embodiments to add textual content to a page. The display 500 is divided into a creator UI column 501 and a graph diagram column 502. The creator UI column includes a pane 510 that in turn includes a name for the page 511; a selectable type 512 of the page; a content entry area 514 for entering content for the page; and formatting controls 513 for formatting parts of the content, such as text in the content. The graph diagram column shows a graph diagram visualization of the in-construction analytic method. In some embodiments, the graph diagram shown is a directed acyclic graph. Presently the graph diagram includes only a single starting node 580 for the starting page of the analytic method, at which the analytic method will begin each time it is applied by a consumer.

While FIG. 5 and each of the display diagrams discussed below show a display whose formatting, organization, informational density, etc., is best suited to certain types of display devices, those skilled in the art will appreciate that actual displays presented by the facility may differ from those shown, in that they may be optimized for particular other display devices or device sizes or other device capabilities, or have shown visual elements omitted, visual elements not shown included, visual elements reorganized, reformatted, revisualized, or shown at different levels of magnification, etc.

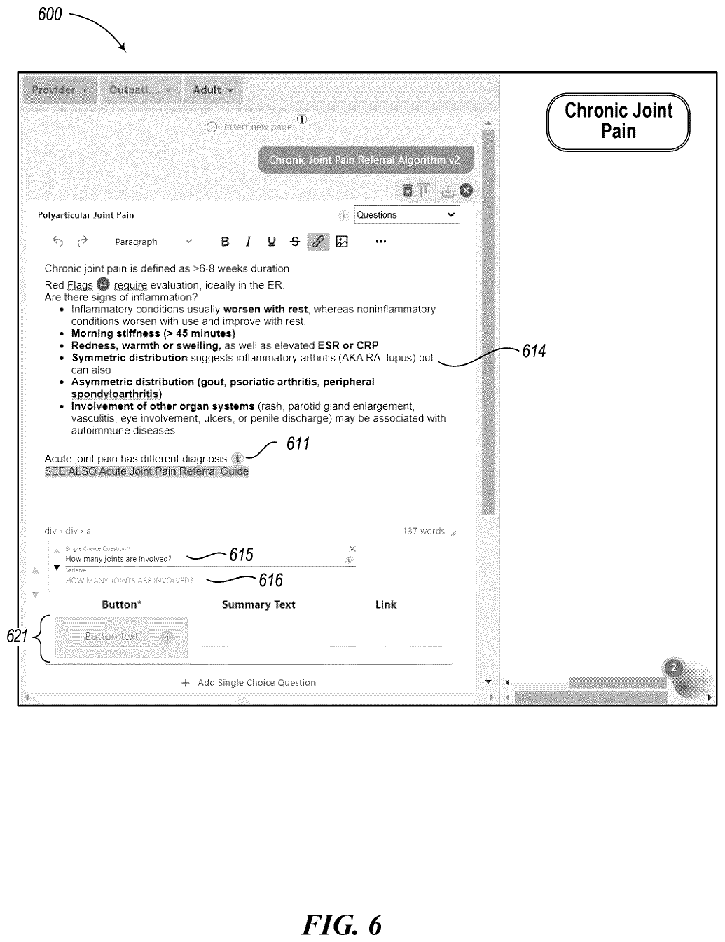

FIG. 6 is a display diagram showing a sample display presented by the facility in some embodiments to enter information about a question to be posed by an analytic method page. The display 600 that the user has entered content 614 for the page. Also, the creator has used a control provided by the facility to insert a supplemental information icon 611 in this content, and enter the following supplemental information text relating to the portion of the content where the creator placed the icon: “including septic, viral (COVID, Parvo), lyme, RMSF, Acute onset, fever, rash, arthritis, must r/o infectious diseases.” The display includes control 615 for specifying the question, here “How many joints are involved?” The display also includes a field 616 into which the facility has automatically populated a variable name whose value during application of the analytic method will be the consumer's answer to this question; the user can edit this prepopulated variable name. The display also includes an expandable area 621 into which the user can enter information about each of the possible answers to this question.

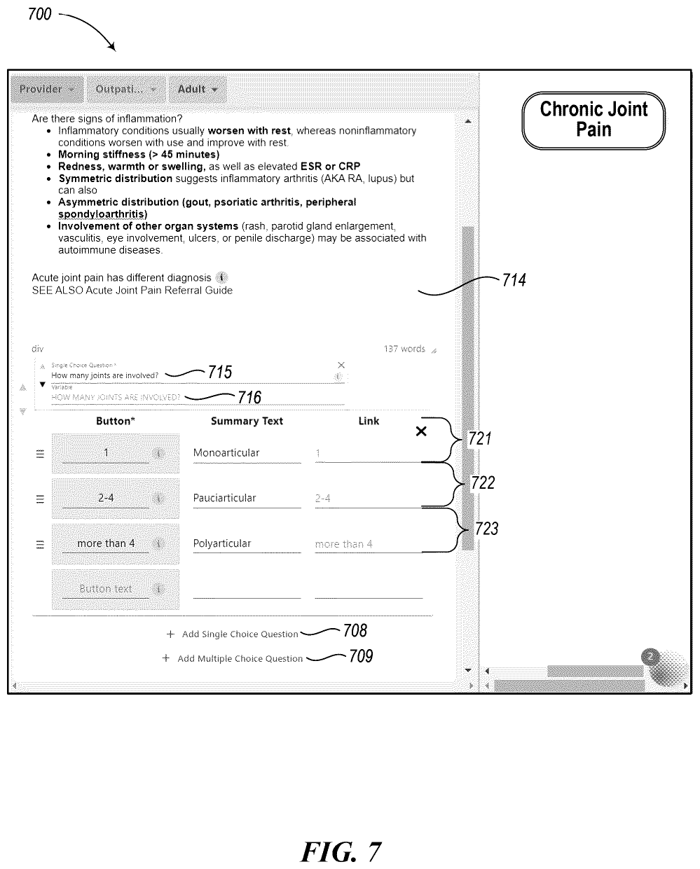

FIG. 7 is a display diagram showing a sample display presented by the facility in some embodiments to define possible answers to a question. The display 700 shows that the user has populated three answers to question 725, answers 721, 722, and 723. For each of the answers, from left to right, the creator has populated button text—“1” for the first button—summary text—“monoarticular” for the first button—and link. The link specifies a routing target when this answer is selected by the consumer. Here, the creator has not entered routing targets, anticipating the later use of a swimlane to route flow based upon the answer to this question in combination with the answer to a following question that the creator is about to establish, by clicking control 708 to add another single choice question to the page.

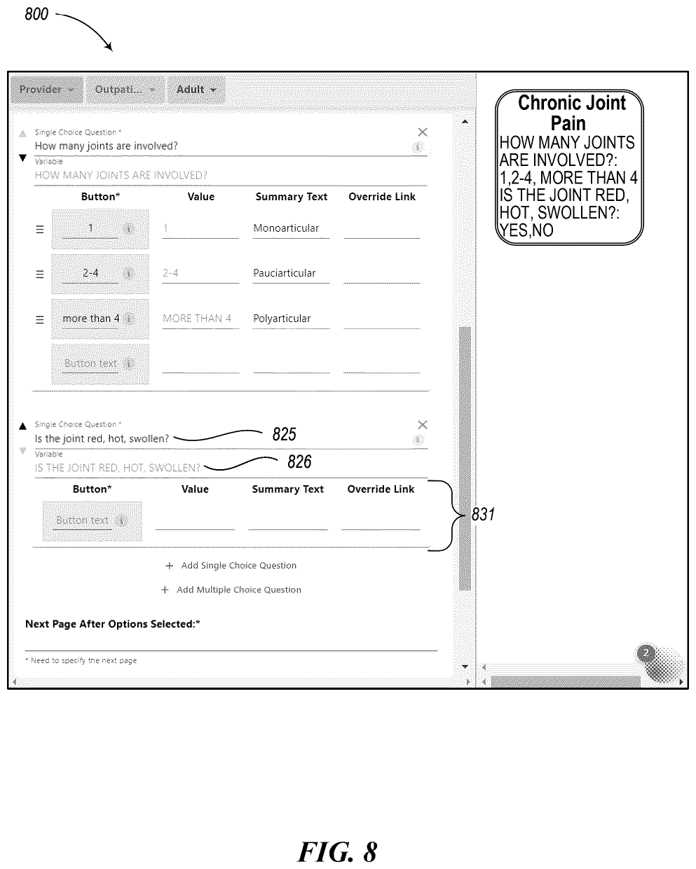

FIG. 8 is a display diagram showing a sample display presented by the facility in some embodiments to define an additional question for the sample analytic method's starting page. It can be seen that, in response to the creator's selection of the add single choice question control 708 shown in FIG. 7, the facility has displayed fields 825 and 826. The creator has entered text “Is the joint red, hot, swollen?” into the question text field 825. The facility has automatically populated variable field 826 with a corresponding variable name. The facility has further displayed section 831 for entering the possible values to this second question.

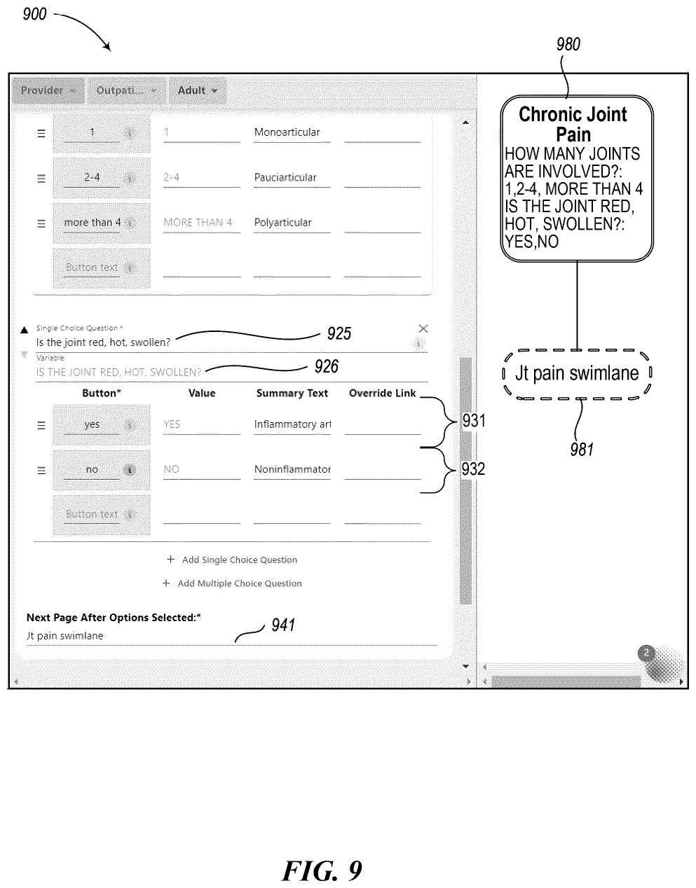

FIG. 9 is a display diagram showing a sample display presented by the facility in some embodiments to enter possible answers to the second question. The creator has populated a “yes” possible answer 931 and a “no” possible answer 932, including summary text for these possible answers. As the result of this input by the creator, for example, if the consumer presses the yes button, the variable “IS THE JOINT RED, HOT, SWOLLEN?” will be set to the value “YES”. The user has also populated a field 941 to specify that flow should proceed from the first page to a “Jt pain swimlane” page. In response, the facility has added a node 981 to the graph diagram corresponding to this new page. It has colored new node 981 red to indicate that its definition as part of the analytic method is not yet complete.

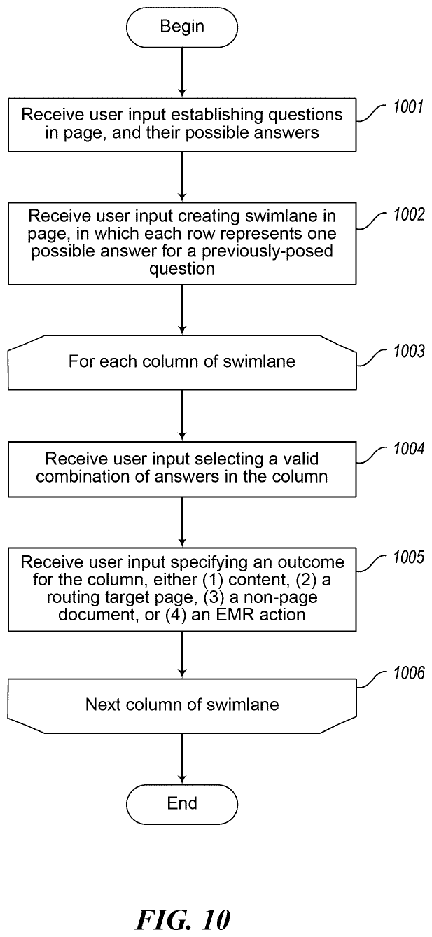

FIG. 10 is a flow diagram showing a process performed by the facility in some embodiments to include a swimlane in an analytic method to specify routing based upon the answers to multiple questions. In step 1001, the facility receives user input establishing questions in a page, and their possible answers. Act 1001 is illustrated in FIGS. 4-9. In act 1002, the facility receives user input creating a swimlane in a page. In the swimlane, each row represents one possible answer for one of the questions previously posted, either in the current page or a page that would be traversed before the current page. In acts 1003-1006, the facility loops through each column of the swimlane. In act 1004, the facility receives user input selecting a valid combination of answers in the column. In act 1005, the facility receives user input specifying an outcome for the column, either content, a target page to route to, a non-page document to display, or an EMR action to take with respect to the patient. In act 1006, if additional columns are to be added to the swimlane, the facility continues in act 1003 to add the next column. After act 1006, this process concludes.

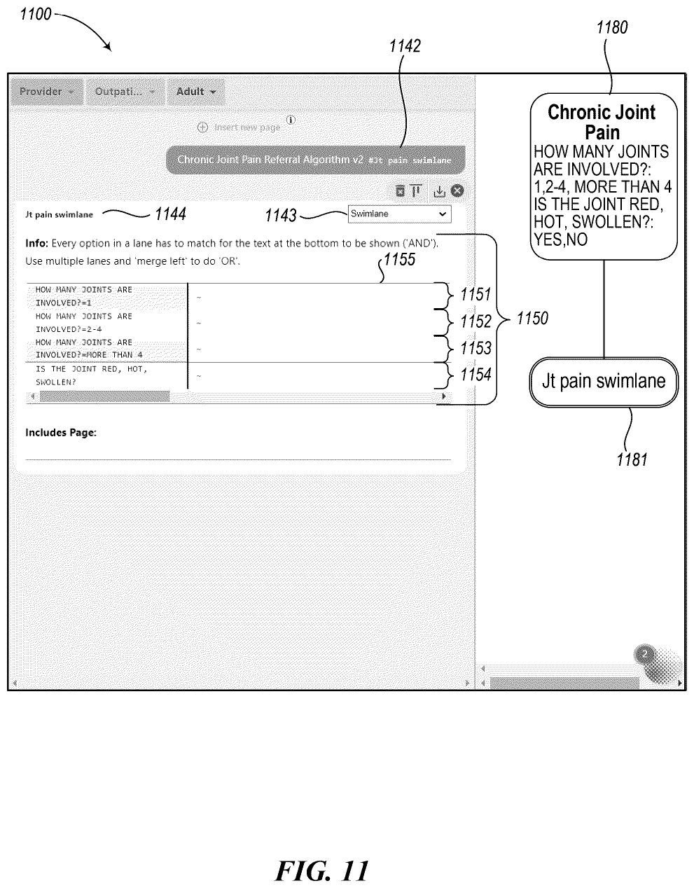

FIG. 11 is a display diagram showing a sample display presented by the facility in some embodiments to begin definition of a swimlane. The display 1100 shows the name 1142 of the new page that will contain the swimlane. The highlighting of graph diagram node 1181 shows that the Jt pain swimlane page is the current page in the creator UI. The display further includes a selection 1143 of a swimlane control; a name 1144 of the swimlane, and rows 1151-1154 of the swimlane each usable to specify a particular answer to one of the questions, i.e., a particular value of the variable defined for one of the questions. The facility is ready for the creator to define a first column 1155 of the swimlane. In particular, in this column, the creator can: select the first row 1151 to specify that the combination of answers includes that the answer to the number of joints question is 1; select the second row 1152 to specify that the answer to the number of joints question is 2-4; select the third row 1153 to specify that the answer to the number of joints question is more than 4, and select the last row 1154 once to specify that the answer to the swollen question is yes, and select the last row twice to indicate that the answer to the swollen question is no. In some embodiments, the facility permits a creator to select a point in a row multiple times to cycle through different possible answers for the question to which the row singularly corresponds. For example, by clicking a first time in row 1154, the creator specifies the answer yes for the corresponding question, which is indicated by a green checkmark. By selecting that point twice, the creator specifies the value no for this question. By clicking a third time, the creator returns to specifying no value for this question. After this point, the cycle repeats.

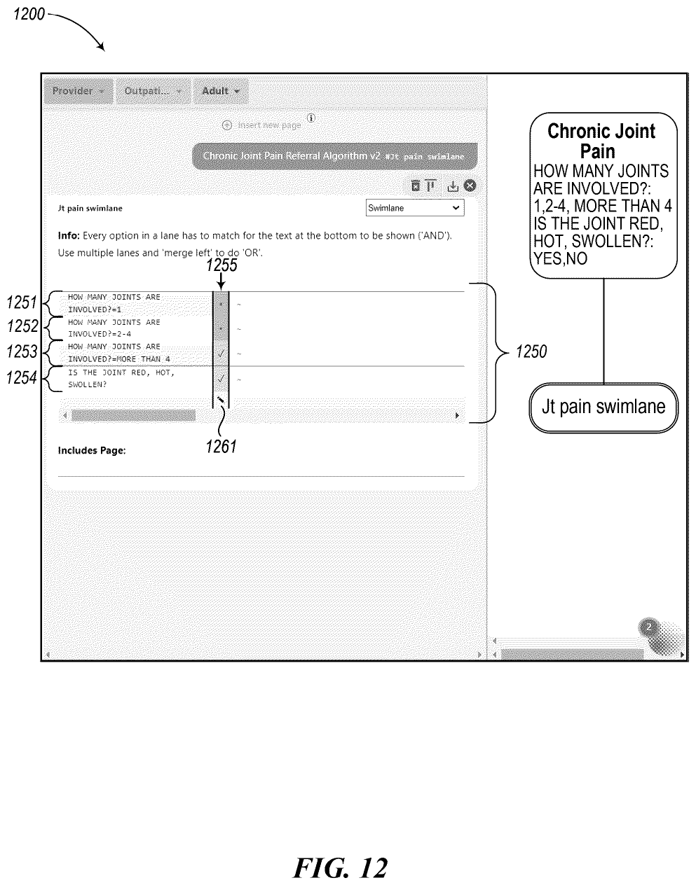

FIG. 12 is a display diagram showing a sample display presented by the facility in some embodiments to define a first column of a swimlane. The display shows that, for first column 1255, the creator has selected among rows 1251-1253 row 1253 to specify that, in the combination of answers representing column 1255, the number of joints question is answered with the more than 4 answer. The creator has also selected row 1254 once to indicate that the answer to the swollen question is yes, represented by a green checkmark. If the creator wanted to specify that the answer to the swollen question is no, the creator would select row 1254 again, changing the green checkmark to a red X.

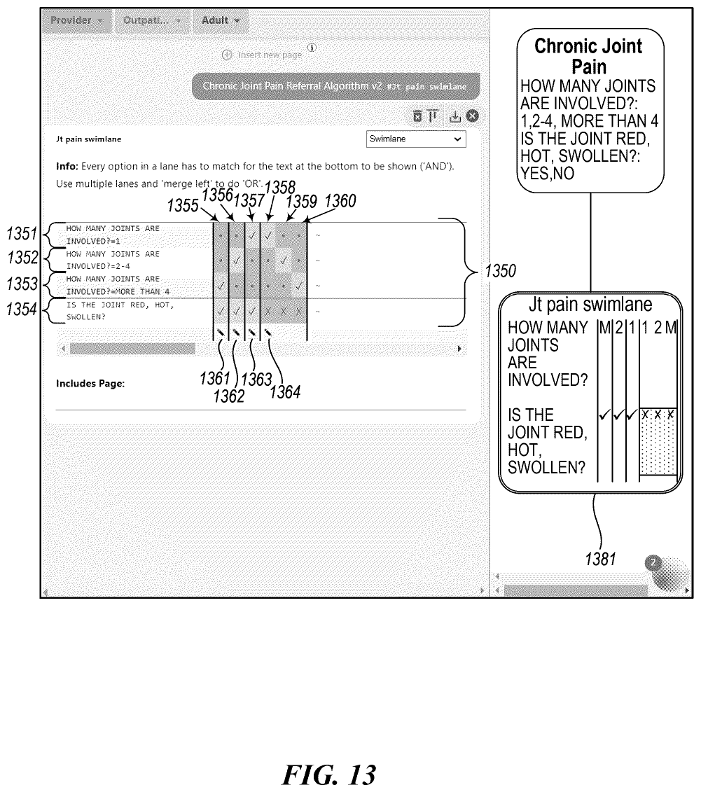

FIG. 13 is a display diagram showing a sample display presented by the facility in some embodiments to define additional columns in a swimlane. By comparing display 1300 shown in FIG. 13 to display 1200 shown in FIG. 12, it can be seen that the creator has added additional swimlane columns 1356-1360 to represent additional combinations of answers to the number of joints and swollen questions. Additionally, the facility displays at the bottom of these columns controls 61-64 for specifying routing targets for these columns. The user can select one of these controls in order to specify the routing target for the combination of answers represented by the column that it is in. For example, the user can select control 1361 to specify routing for the combination of answering the number of joints question with answer more than 4, and answering the swollen question with yes.

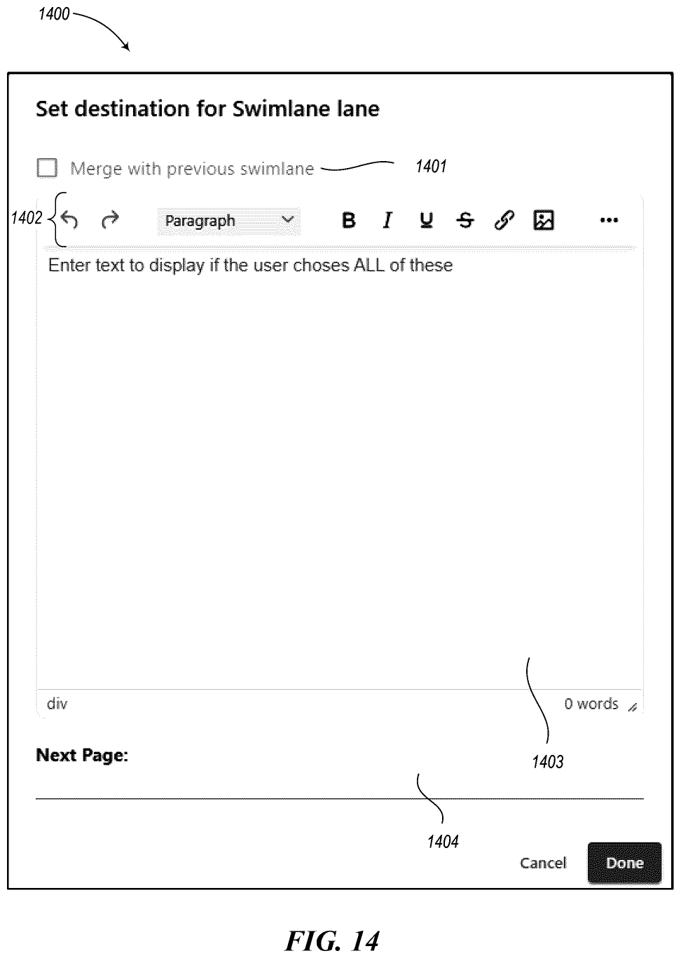

FIG. 14 is a display diagram showing a sample display presented by the facility in some embodiments to specify the routing target for a particular swimlane column. In some embodiments, the facility presents display 1400 as a pop-up window over the creator user interface. The display 1400 includes a checkbox 1401 that the user can select in order to merge this column with the column to the left, if there is one. Where the creator does this, the creator need not enter any routing information, as the routing target specified for or reused by the column to the left will be used. The display also includes a field 1403 into which the creator can enter content, such as text, to be displayed in response to the consumer submitting the answers to which the swimlane column corresponds. The display also includes a field 1404 where the creator can specify a name of a different page to which the consumer should be routed when the combination of answers to which the swimlane column corresponds are submitted.

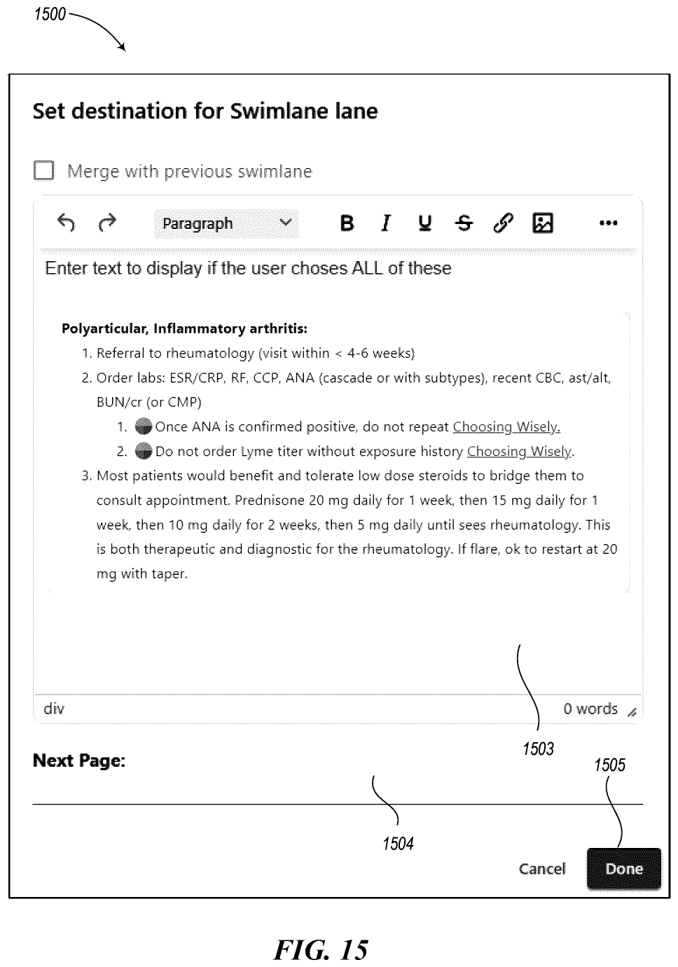

FIG. 15 is a display diagram showing a sample display presented by the facility in some embodiments to enter content to display if a consumer submits the combination of answers to which the leftmost swimlane column corresponds. It can be seen that the user has entered content into field 1503 to be displayed to consumers that enter the combination of answers to which the swimlane column corresponds. After entering this content, the creator selects a done control 1505 to dismiss the pop-up.

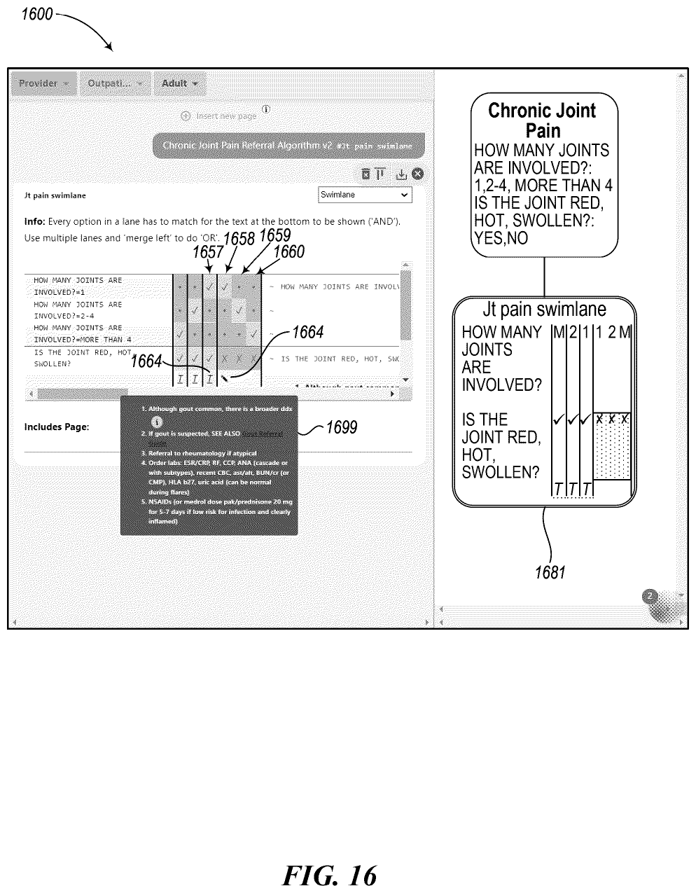

FIG. 16 is a display diagram showing a sample display presented by the facility in some embodiments to display content previously defined for a swimlane column. In the display, can be seen that, in response to the creator hovering over routing target control 1663 for the third swimlane column 1657 the facility has displayed a compact pop-up window 1699 containing the content earlier specified by the creator for this column. At this point, the creator can select routing target control 1664 to specify a routing target for columns 1658-1660, which have previously been grouped by an action of the creator.

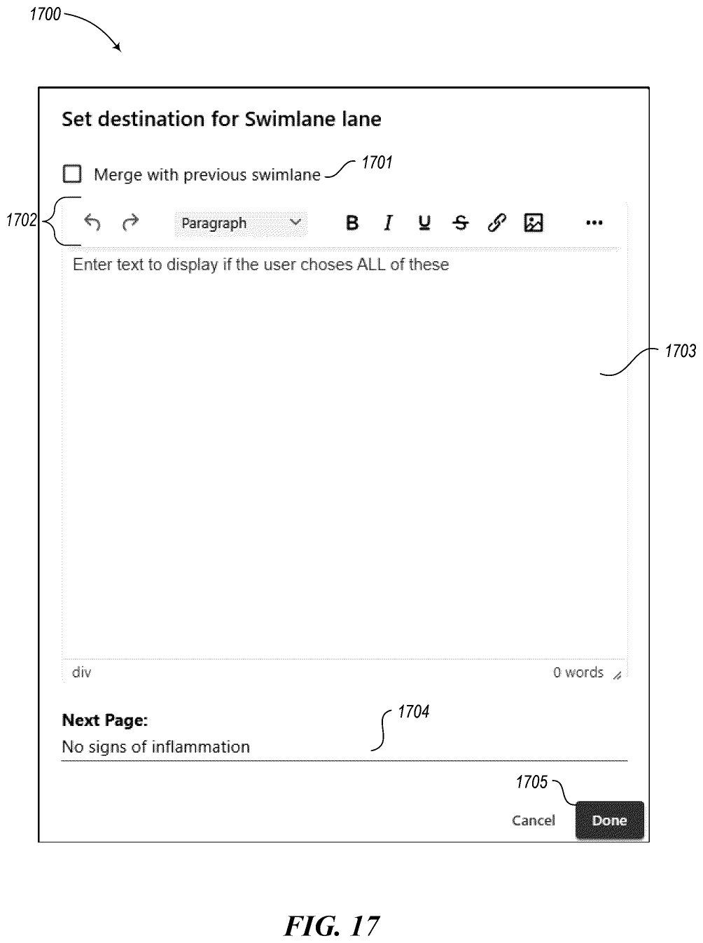

FIG. 17 is a display diagram showing a sample display presented by the facility in some embodiments to specify a routing target for the fourth through sixth columns of the swimlane. It can be seen that the creator has entered the page name “No signs of inflammation” into next page field 1704. The creator selects the done control 1705 to close display 1700.

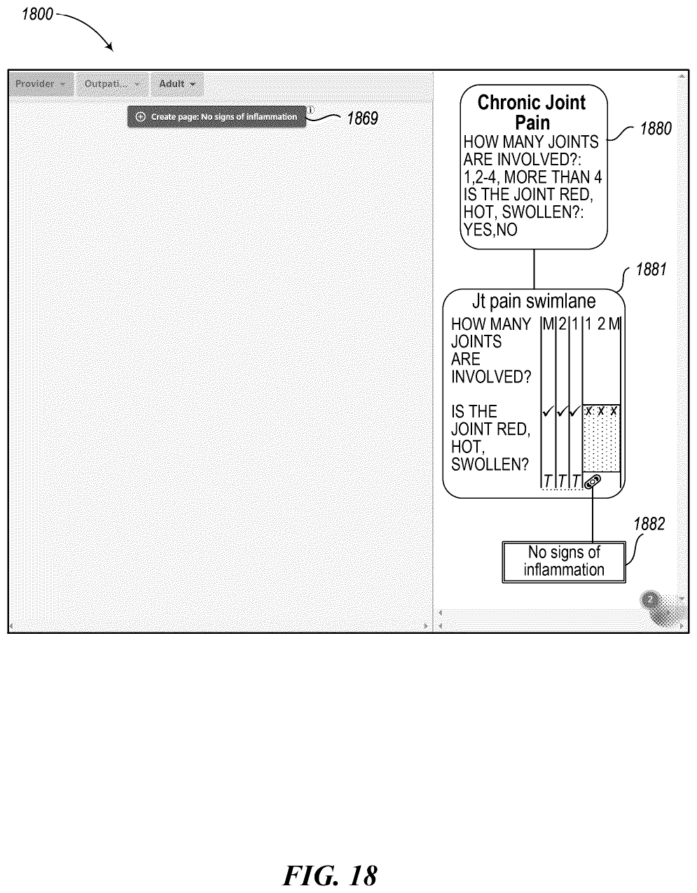

FIG. 18 is a display diagram showing a sample display presented by the facility in some embodiments to create a new page that was specified as the target of the fourth through sixth columns of the swimlane. It can be seen that the facility has added node 1881 to the graph diagram representing the completed swimlane. In some embodiments, the creator can hover over the routing target icons at the bottom of the node to see the routing target specified for each of the swimlane columns. Additionally, the facility has added a placeholder node 1882 for the No Signs of Inflammation page implicated by the routing target specified for the fourth through sixth columns of the swimlane. The user can activate a control 1869 to create this page.

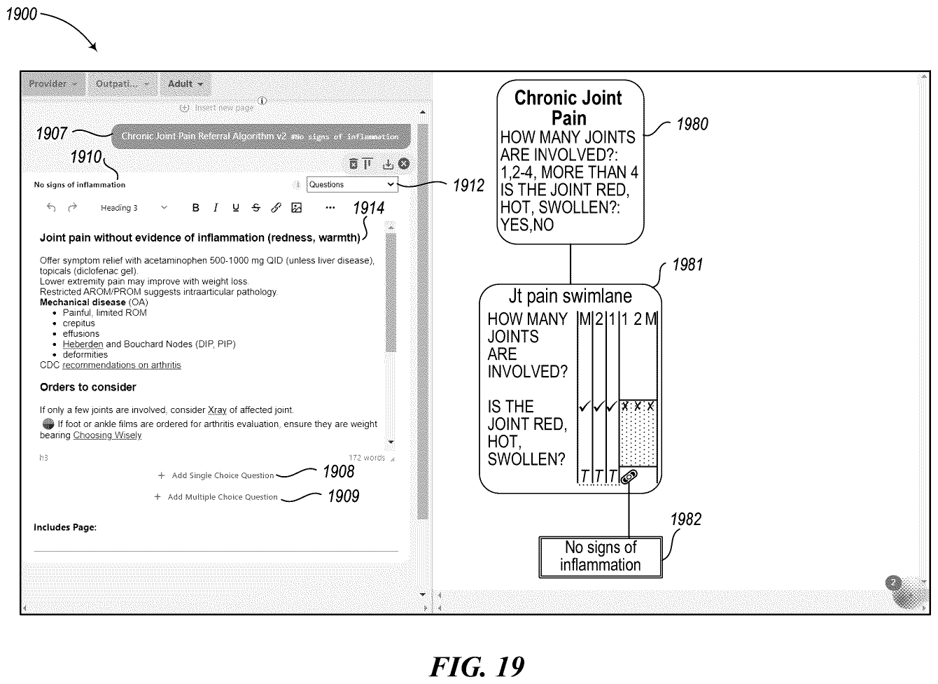

FIG. 19 is a display diagram showing a sample display presented by the facility in some embodiments to enter content for the no signs of inflammation page. The creator has specified a questions type 1912 for this page, and entered content for the page into content field 1914. At this point, the facility has replaced the placeholder node in the graph diagram with normal node 1982, reflecting that the no signs of inflammation page has been adequately defined. The user can add a single choice question to this page by activating control 1908.

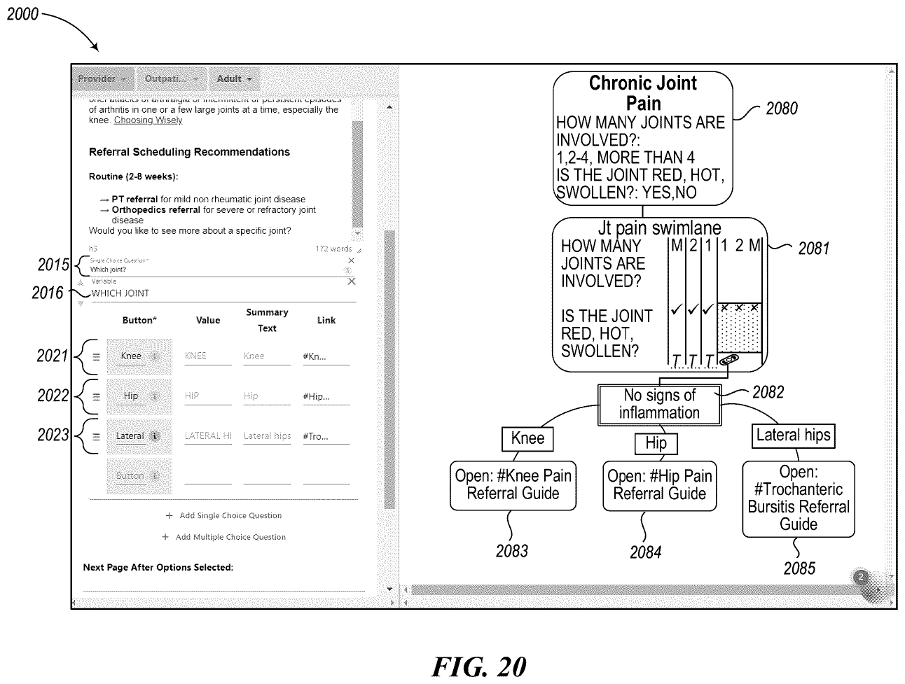

FIG. 20 is a display diagram showing a sample display presented by the facility in some embodiments to add a question to the no signs of inflammation page. It can be seen that the creator has added a “Which joint?” question 2015 to this page, for which the facility has populated a suggested variable name into variable field 2016. The creator has also specified answers 2021-2023 for this question as part of defining these answers, the creator has specified in the link column the name of a different document external to the analytic methods for each answer. For example, though not fully visible, the creator has added in the link column for answer 2021 #KneePainReferralGuide. In response to entering this information, the facility has created new nodes 2083-2085 in the graph diagram, each for corresponding to an external document specified for a different one of the three answers. For example, node 2083 corresponds to answer 2021. The green halo around node 2082 in the graph diagram reflects that the corresponding page of the analytic method is presently being defined or edited in the creator user interface to the left.

Table 1 below shows an internal representation generated by the facility in JSON for the example analytic method whose creation is shown in FIGS. 4-20 and discussed above.

TABLE 1

| | |
|---|---|
| 1 | { |
| 2 | ″data″: { |
| 3 | ″id″: ″7e7f5e2b-9722-4dbc-a32a-9c8e465a033a″, |
| 4 | ″state″: ″DRAFT″, |
| 5 | ″title″: ″Chronic Joint Pain Referral Algorithm |
| | v2″, |
| 6 | ″tags″: [ |
| 7 | ″chronic joint issues″, |
| 8 | ″chronic joint pain″, |
| 9 | ″joint pain″, |
| 10 | ″monoarticular joint pain″, |
| 11 | ″multiple joints″, |
| 12 | ″pain in joint″, |
| 13 | ″pauciarticular joint pain″, |
| 14 | ″polyarticular joint pain″, |
| 15 | ″psoriatric arthritis″, |
| 16 | ″spondyloarthritis″ |
| 17 | ], |
| 18 | ″topic_subtopic_pairs″: [ ], |
| 19 | ″docCategories″: [ |
| 20 | { |
| 21 | ″id″: ″b0e2f7fc-1be9-4ea2-b80b- |
| | cd2b57b73bdf″, |
| 22 | ″name″: ″Orthopedic Surgery″ |
| 23 | }, |
| 24 | { |
| 25 | ″id″: ″f2d4ed1f-0785-48ba-9238- |
| | 9c9f267be053″, |
| 26 | ″name″: ″Pharmacy″ |
| 27 | }, |
| 28 | { |
| 29 | ″id″: ″1b49d385-563a-4dc7-98cb- |
| | 7e02fbf4e4bf″, |
| 30 | ″name″: ″Rheumatology″ |
| 31 | }, |
| 32 | { |
| 33 | ″id″: ″af3d0c73-48c9-495c-b8f2- |
| | ee843187a6f8″, |
| 34 | ″name″: ″Sports Medicine″ |
| 35 | }, |
| 36 | { |
| 37 | ″id″: ″59c4aace-0a34-4d67-b967- |
| | fb4393cd16a9″, |
| 38 | ″name″: ″Non-Commercial″ |
| 39 | } |
| 40 | ], |
| 41 | ″docCredits″: [ |
| 42 | { |
| 43 | ″title″: ″Contributor″, |
| 44 | ″name″: ″Sue Chung, MD - Rheumatology″ |
| 45 | }, |
| 46 | { |
| 47 | ″title″: ″Contributor″, |
| 48 | ″name″: ″Rebecca Poage″ |
| 49 | }, |
| 50 | { |
| 51 | ″title″: ″Published by″, |
| 52 | ″name″: ″Pearl Editorial Team″ |
| 53 | }, |
| 54 | { |
| 55 | ″title″: ″Last update″, |
| 56 | ″name″: ″2022-06-24T14:34:09.000Z″ |
| 57 | }, |
| 58 | { |
| 59 | ″title″: ″Next review″, |
| 60 | ″name″: ″2026-01-05T19:04:20.961Z″ |
| 61 | } |
| 62 | ], |
| 63 | ″governanceModel″: { |
| 64 | ″id″: ″3ce49eff-855f-4226-b736- |
| | 232dd137ac6e″, |
| 65 | ″name″: ″Clinical Expertise″ |
| 66 | }, |
| 67 | ″document″: { |
| 68 | ″id″: ″b80548eb-c2cb-4cea-9a44- |
| | e0f801a054d6″, |
| 69 | ″organization″: { |
| 70 | ″id″: ″be3d0552-47e8-403b-9783- |
| | 4d2d3e1419c5″ |
| 71 | }, |
| 72 | ″docType″: ″DOCUMENT″, |
| 73 | ″members″: [ |
| 74 | { |
| 75 | ″userId″: ″17e97550-fa72-45bc- |
| | a2fa-f3979e071342″, |
| 76 | ″name″: ″Eve Cunningham″, |
| 77 | ″role″: ″CONTRIBUTOR″ |
| 78 | }, |
| 79 | { |
| 80 | ″userId″: ″2a865e31-cc50-4a03- |
| | 9776-1b79b15eaf3f″, |
| 81 | ″name″: ″Steve Lawrenz″, |
| 82 | ″role″: ″OWNER″ |
| 83 | } |
| 84 | ] |
| 85 | }, |
| 86 | ″hasAlgorithm″: null, |
| 87 | ″hasGuides″: null, |
| 88 | ″nextReviewAt″: ″2026-01-05″, |
| 89 | ″createdAt″: ″2022-07-05T18:04:41.879Z″, |
| 90 | ″updatedAt″: ″2022-07-09T03:27:41.839Z″, |
| 91 | ″pages″: [ |
| 92 | { |
| 93 | ″id″: ″3d08659f-cc1b-45c8-b775- |
| | b01745e6019e″, |
| 94 | ″isGuide″: false, |
| 95 | ″isRoot″: true, |
| 96 | ″persona″: { |
| 97 | ″id″: ″0c82dbce-1000-4c54-ad20- |
| | 18af4f383bdb″, |
| 98 | ″name″: ″Provider″ |
| 99 | }, |
| 100 | ″careCategory″: { |
| 101 | ″id″: ″4c6c897d-70de-4e4a-9a21- |
| | 15e99cf4b444″, |
| 102 | ″name″: ″Outpatient″ |
| 103 | }, |
| 104 | ″patientGroup″: { |
| 105 | ″id″: ″59b96b3d-d330-418d-8054- |
| | 773f5b3fb24c″, |
| 106 | ″name″: ″Adult″ |
| 107 | }, |
| 108 | ″name″: ″Chronic Joint Pain″, |
| 109 | ″content″: { |
| 110 | ″content″: ″\u003cdiv\u003eChronic |
| | joint pain is defined as |
| | \u0026gt;6-8 weeks |
| | duration.\u003c/div\u003e\n\u003cd |
| | iv\u003eRedFlags\u003caction |
| | id=\″4358307549704576\″ |
| | class=\″actionNonEditable\″ |
| | title=\″Red |
| | Flag\″type=\″redflag\″\u003e\u003c |
| | actioncontentclass=\″actionNonEdit |
| | able\″ |
| | type=\″redflag\″\u003e\n\u003cdiv\ |
| | u003eFever\u003c/div\u003e\n\u003c |
| | div\u003eConcern for |
| | septicarthritis\u003c/div\u003e\n\ |
| | u003cdiv\u003eSudden onset, warm, |
| | inability to move |
| | joint\u003c/div\u003e\n\u003cdiv\u |
| | 003eHypotension\u003c/div\u003e\n\ |
| | u003cdiv\u003eTachycardia\u003c/di |
| | v\u003e\n\u003c/actioncontent\u003 |
| | e\u003c/action\u003erequire |
| | evaluation, ideally in the |
| | ER.\u003c/div\u003e\n\u003cdiv\u00 |
| | 3e\n\u003cdiv\u003eAre there signs |
| | of |
| | inflammation?\u003c/div\u003e\n\u0 |
| | 03cdiv\u003e\n\u003cul\u003e\n\u00 |
| | 3cli\u003eInflammatory conditions |
| | usually\u003cstrong\u003eworsen |
| | with rest\u003c/strong\u003e, |
| | whereas noninflammatoryconditions |
| | worsen with use and improve with |
| | rest.\u003c/li\u003e\n\u003cli\u00 |
| | 3e\u003cstrong\u003eMorning |
| | stiffness (\u0026gt; 45 |
| | minutes)\u003c/strong\u003e\u003c/ |
| | li\u003e\n\u003cli\u003e\u003cstro |
| | ng\u003eRedness, warmth or |
| | swelling,\u003c/strong\u003e as |
| | well as elevated |
| | \u003cstrong\u003eESR or |
| | CRP\u003c/strong\u003e\u003c/li\u0 |
| | 03e\n\u003cli\u003e\u003cstrong\u0 |
| | 03eSymmetric |
| | distribution\u003c/strong\u003esug |
| | gests inflammatory arthritis (AKA |
| | RA, lupus) but can |
| | also\u0026nbsp;\u003c/li\u003e\n\u |
| | 003cli\u003e\u003cstrong\u003eAsym |
| | metric\u003c/strong\u003e\u003cstr |
| | ong\u003edistribution (gout, |
| | psoriatic arthritis, peripheral |
| | spondyloarthritis)\u003c/strong\u0 |
| | 03e\u003c/li\u003e\n\u003cli\u003e |
| | \u003cstrong\u003eInvolvement of |
| | other organ |
| | systems\u003c/strong\u003e (rash, |
| | parotid gland enlargement, |
| | vasculitis, eye involvement, |
| | ulcers, or penile discharge) may |
| | be associated with autoimmune |
| | diseases.\u0026nbsp;\u003c/li\u003 |
| | e\n\u003c/ul\u003e\n\u003c/div\u00 |
| | 3e\n\u003c/div\u003e\n\u003cdiv\u0 |
| | 03e\u003c/div\u003e\n\u003cdiv\u00 |
| | 3eAcute joint pain has different |
| | diagnosis\u003caction |
| | id=\″501863142383346\″ |
| | class=\″actionNonEditable\″ |
| | title=\″Info\″type=\″info\″\u003e\ |
| | u003cactioncontentclass=\″actionNo |
| | nEditable\″type=\″info\″\u003eincl |
| | uding septic, viral (COVID, |
| | Parvo), lyme, RMSF. Acute onset, |
| | fever, rash, arthritis, must r/o |
| | infectious |
| | diseases.\u003c/actioncontent\u003 |
| | e\u003c/action\u003e\n\u003cdiv\u0 |
| | 03e\u003ca class=\″internal- |
| | link\″href=\″#Acute%20Joint%20Pain |
| | %20Referral%20Guide\″target=\″_sel |
| | f\″\u003eSEE ALSO Acute Joint Pain |
| | Referral |
| | Guide\u003c/a\u003e\u003c/div\u003 |
| | e\n\u003cdiv\u003e\u003c/div\u003e |
| | \n\u003c/div\u003e″, |
| 111 | ″tooltip″: ″″, |
| 112 | ″pageType″: ″multiple_questions″, |
| 113 | ″next_link″: ″Jt pain swimlane″, |
| 114 | ″questions″: [ |
| 115 | { |
| 116 | ″id″: |
| | ″8381636892077351″, |
| 117 | ″type″: ″exclusive″, |
| 118 | ″options″: [ |
| 119 | { |
| 120 | ″id″: |
| | ″4283276934 |
| | 121146″, |
| 121 | ″text″: ″1″, |
| 122 | ″summary |
| | text″: |
| | ″Monoarticu |
| | lar″ |
| 123 | }, |
| 124 | { |
| 125 | ″id″: |
| | ″32456561004 |
| | 96640″, |
| 126 | ″text″: ″2- |
| | 4″, |
| 127 | ″tooltip″: |
| | ″″, |
| 128 | ″summary |
| | text″: |
| | ″Pauciarticu |
| | lar″ |
| 129 | }, |
| 130 | { |
| 131 | ″id″: |
| | ″43745212045 |
| | 63388″, |
| 132 | ″text″: |
| | ″more than |
| | 4″, |
| 133 | ″summary |
| | text″: |
| | ″Polyarticul |
| | ar″ |
| 134 | } |
| 135 | ], |
| 136 | ″question″: ″How many |
| | joints are involved?″ |
| 137 | }, |
| 138 | { |
| 139 | ″id″: |
| | ″3077669910798072″, |
| 140 | ″type″: ″exclusive″, |
| 141 | ″options″: [ |
| 142 | { |
| 143 | ″id″: |
| | ″37400030347 |
| | 28790″, |
| 144 | ″text″: |
| | ″yes″, |
| 145 | ″summary |
| | text″: |
| | ″Inflammator |
| | y arthritis″ |
| 146 | }, |
| 147 | { |
| 148 | ″id″: |
| | ″21228283940 |
| | 27450″, |
| 149 | ″text″: |
| | ″no″, |
| 150 | ″tooltip″: |
| | ″\u003cdiv\u |
| | 003eSwelling |
| | alone does |
| | not suggest |
| | inflammatory |
| | arthritis.\u |
| | 0026nbsp;\u0 |
| | 026nbsp;\u00 |
| | 3c/div\u003e |
| | ″, |
| 151 | ″summary |
| | text″: |
| | ″Noninflamma |
| | tory |
| | arthritis″ |
| 152 | } |
| 153 | ], |
| 154 | ″question″: ″Is the |
| | joint red, hot, |
| | swollen?″ |
| 155 | } |
| 156 | ] |
| 157 | }, |
| 158 | ″createdAt″: ″2022-07- |
| | 05T18:04:41.906Z″, |
| 159 | ″updatedAt″: ″2022-07-05T18:04:41.906Z″ |
| 160 | }, |
| 161 | { |
| 162 | ″id″: ″c6d7354a-d20a-4963-a7e9- |
| | 28202a72a8bb″, |
| 163 | ″isGuide″: false, |
| 164 | ″isRoot″: false, |
| 165 | ″persona″: { |
| 166 | ″id″: ″0c82dbce-1000-4c54-ad20- |
| | 18af4f383bdb″, |
| 167 | ″name″: ″Provider″ |
| 168 | }, |
| 169 | ″careCategory″: { |
| 170 | ″id″: ″4c6c897d-70de-4e4a-9a21- |
| | 15e99cf4b444″, |
| 171 | ″name″: ″Outpatient″ |
| 172 | }, |
| 173 | ″patientGroup″: { |
| 174 | ″id″: ″59b96b3d-d330-418d-8054- |
| | 773f5b3fb24c″, |
| 175 | ″name″: ″Adult″ |
| 176 | }, |
| 177 | ″name″: ″Jt pain swimlane″, |
| 178 | ″content″: { |
| 179 | ″lanes″: [ |
| 180 | { |
| 181 | ″id″: ″f07a7e6d-f5f5- |
| | 4848-a9a1- |
| | f8ccb5660fb6″, |
| 182 | ″link″: ″″, |
| 183 | ″choices″: { |
| 184 | ″HOW MANY JOINTS |
| | ARE INVOLVED?″: |
| | ″MORE THAN 4″, |
| 185 | ″IS THE JOINT RED, |
| | HOT, SWOLLEN?″: |
| | ″YES″ |
| 186 | }, |
| 187 | ″content″: |
| | ″\u003cdiv\u003e\u003c/ |
| | div\u003e\n\u003col\u00 |
| | 3e\n\u003cli\u003eRefer |
| | ral to rheumatology |
| | (visit within \u0026lt; |
| | 4-6 |
| | weeks)\u003c/li\u003e\n |
| | \u003cli\u003eOrder |
| | labs: ESR/CRP, RF, CCP, |
| | ANA (cascade or with |
| | subtypes), recent CBC, |
| | ast/alt, BUN/cr (or |
| | CMP)\n\u003col\u003e\n\ |
| | u003cli\u003e\u003c |
| | action |
| | class=\″actionNonEditab |
| | le\″ |
| | type=\″choosingwisely\″ |
| | \u003e\u003c/action |
| | \u003eOnce ANA is |
| | confirmed positive, do |
| | not repeat \u003ca |
| | class=\″external-link\″ |
| | href=\″ |
| | https://www.choosingwis |
| | ely.org/clinician- |
| | lists/american-college- |
| | rheumatology- |
| | repeatconfirmed- |
| | positive-ana-in- |
| | patients-with-jia-or- |
| | systemic-lupus/\″ |
| | target=\″_blank\″ |
| | rel=\″noopener\″\u003eC |
| | hoosing |
| | Wisely.\u003c/a\u003e\u |
| | 003c/li\u003e\n\u003cli |
| | \u003e\u003caction |
| | id=\″3531289296128592\″ |
| | class=\″actionNonEditab |
| | le\″title=\″Choosing |
| | Wisely\″ |
| | type=\″choosingwisely\″ |
| | \u003e\u003c/action\u00 |
| | 3eDo not order Lyme |
| | titer without exposure |
| | history\u0026nbsp;\u003 |
| | ca class=\″external- |
| | link\″href=\″https://ww |
| | w.choosingwisely.org/so |
| | cieties/american- |
| | college-of- |
| | rheumatology-pediatric- |
| | rheumatology/\″ |
| | target=\″_blank\″rel=\″ |
| | noopener\″\u003eChoosin |
| | gWisely\u003c/a\u003e.\ |
| | u003c/li\u003e\n\u003c/ |
| | ol\u003e\n\u003c/li\u00 |
| | 3e\n\u003cli\u003eMost |
| | patients would benefit |
| | and tolerate low dose |
| | steroids to bridge them |
| | to consult appointment. |
| | Prednisone 20 mg daily |
| | for 1 week, then 15 mg |
| | daily for 1 week, then |
| | 10 mg daily for 2 |
| | weeks, then 5 mg daily |
| | until sees |
| | rheumatology. This is |
| | both therapeutic and |
| | diagnostic for the |
| | rheumatology. If flare, |
| | ok to restart at 20 mg |
| | with |
| | taper.\u003c/li\u003e\n |
| | \u003c/ol\u003e″, |
| 188 | ″mergeLeft″: false |
| 189 | }, |
| 190 | { |
| 191 | ″id″: ″f84a8032-4612- |
| | 4c12-8809- |
| | 0d45c507638b″, |
| 192 | ″link″: ″″, |
| 193 | ″choices″: { |
| 194 | ″HOW MANY JOINTS |
| | ARE INVOLVED?″: |
| | ″2-4″, |
| 195 | ″IS THE JOINT |
| | RED, HOT, |
| | SWOLLEN?″: ″YES″ |
| 196 | }, |
| 197 | ″content″: |
| | ″\u003ch4\u003e\u003cac |
| | tion |
| | id=\″2513088233032590\″ |
| | class=\″actionNonEditab |
| | le\″ |
| | title=\″Info\″type=\″in |
| | fo\″\u003e\u003cactionc |
| | ontent |
| | class=\″actionNonEditab |
| | le\″type=\″info\″\u003e |
| | \n\u003cdiv\u003eDDX |
| | spondyloarthritis, |
| | psoriatic arthritis, |
| | AS\u003c/div\u003e\n\u0 |
| | 03c/actioncontent\u003e |
| | \u003c/action\u003e\u00 |
| | 3c/h4\u003e\n\u003col\u |
| | 003e\n\u003cli\u003eRef |
| | erral to rheumatology |
| | (visit within \u0026lt; |
| | 4-6 |
| | weeks)\u003c/li\u003e\n |
| | \u003cli\u003eOrder |
| | labs: ESR/CRP, RF, CCP, |
| | ANA (cascade or with |
| | subtypes), recent CBC, |
| | ast/alt, BUN/cr (or |
| | CMP), HLA |
| | b27\n\u003cul\u003e\n\u |
| | 003cli\u003e\u003cactio |
| | n |
| | class=\″actionNonEditab |
| | le\″type=\″choosingwise |
| | ly\″\u003e\u003c/action |
| | \u003eOnce ANA is |
| | confirmed positive, do |
| | not repeat \u003ca |
| | class=\″external-link\″ |
| | href=\″https://www.choo |
| | singwisely.org/clinicia |
| | n-lists/american- |
| | college-rheumatology- |
| | repeatconfirmed- |
| | positive-ana-in- |
| | patients-with-jia-or- |
| | systemic-lupus/\″ |
| | target=\″_blank\″ |
| | rel=\″noopener\″\u003eC |
| | hoosingWisely.\u003c/a\ |
| | u003e\u003c/li\u003e\n\ |
| | u003cli\u003e\u003cacti |
| | onid=\″3531289296128592 |
| | \″ |
| | class=\″actionNonEditab |
| | le\″title=\″Choosing |
| | Wisely\″type=\″choosing |
| | wisely\″\u003e\u003c/ac |
| | tion\u003eDo not order |
| | Lyme titer without |
| | exposure |
| | history\u0026nbsp;\u003 |
| | ca class=\″external- |
| | link\″href=\″https://ww |
| | w.choosingwisely.org/so |
| | cieties/american- |
| | college-of- |
| | rheumatology-pediatric- |
| | rheumatology/\″ |
| | target=\″_blank\″ |
| | rel=\″noopener\″\u003eC |
| | hoosingWisely\u003c/a\u |
| | 003e.\u003c/li\u003e\n\ |
| | u003c/ul\u003e\n\u003c/ |
| | li\u003e\n\u003cli\u003 |
| | eSkin exam for nail |
| | pitting and psoriatic |
| | plaques (including |
| | inverse psoriasis on |
| | groin, axilla, scrotum |
| | -shiny and could be |
| | mistaken for tinea. See |
| | photos at \u003ca |
| | class=\″external- |
| | link\″href=\″https://de |
| | rmnetnz.org/topics/flex |
| | ural-psoriasis\″ |
| | target=\″_blank\″ |
| | rel=\″noopener\″\u003eD |
| | ermNetNZ\u003c/a\u003e) |
| | \u003c/li\u003e\n\u003c |
| | li\u003eNSAID dujour to |
| | bridge until |
| | rheumatology visit |
| | (caution with |
| | prednisone, since it |
| | can convert psoriasis |
| | into severe plaque |
| | psoriasis)\u003c/li\u00 |
| | 3e\n\u003c/ol\u003e″, |
| 198 | ″mergeLeft″: false |
| 199 | }, |
| 200 | { |
| 201 | ″id″: ″249437d6-9c0a- |
| | 4bc3-a933- |
| | 45929bdd4f12″, |
| 202 | ″link″: ″″, |
| 203 | ″choices″: { |
| 204 | ″HOW MANY JOINTS |
| | ARE INVOLVED?″: |
| | ″1″, |
| 205 | ″IS THE JOINT |
| | RED, HOT, |
| | SWOLLEN?″: ″YES″ |
| 206 | }, |
| 207 | ″content″: |
| | ″\u003col\u003e\n\u003c |
| | li\u003eAlthough gout |
| | common, there is a |
| | broader ddx\u003caction |
| | id=\″5542419401334181\″ |
| | class=\″actionNonEditab |
| | le\″ title=\″Info\″ |
| | type=\″info\″\u003e\u00 |
| | 3cactioncontentclass=\″ |
| | actionNonEditable\″ |
| | type=\″info\″\u003e\n\u |
| | 003cdiv\u003eDDX gout, |
| | pseudogout, |
| | undifferentiated RA, |
| | psoriatric, |
| | spondylarthritis\u003c/ |
| | div\u003e\n\u003c/actio |
| | ncontent\u003e\u003c/ac |
| | tion\u003e\u003c/li\u00 |
| | 3e\n\u003cli\u003eIf |
| | gout is suspected, SEE |
| | ALSO |
| | \u003caclass=\″internal |
| | -link\″ |
| | href=\″#Gout%20Referral |
| | %20Guide\″ |
| | target=\″_self\″\u003eG |
| | out |
| | ReferralGuide\u003c/a\u |
| | 003e\u003c/li\u003e\n\u |
| | 003cli\u003eReferral to |
| | rheumatology if |
| | atypical\u003c/li\u003e |
| | \n\u003cli\u003eOrder |
| | labs: ESR/CRP, RF, CCP, |
| | ANA (cascade or with |
| | subtypes), recent CBC, |
| | ast/alt, BUN/cr (or |
| | CMP), HLA b27, uric |
| | acid (can be normal |
| | during |
| | flares)\u003c/li\u003e\ |
| | n\u003cli\u003eNSAIDs |
| | (or medrol dose |
| | pak/prednisone 20 mg |
| | for 5-7 days if low |
| | risk for infection and |
| | clearly |
| | inflamed)\u003c/li\u003 |
| | e\n\u003c/ol\u003e\n\u0 |
| | 03cdiv\u003e\u003c/div\ |
| | u003e″, |
| 208 | ″mergeLeft″: false |
| 209 | }, |
| 210 | { |
| 211 | ″id″: ″4c36c843-b276- |
| | 4201-b66d- |
| | 6e3230179d08″, |
| 212 | ″link″: ″No signs of |
| | inflammation″, |
| 213 | ″choices″: { |
| 214 | ″HOW MANY JOINTS |
| | ARE INVOLVED?″: |
| | ″1″, |
| 215 | ″IS THE JOINT |
| | RED, HOT, |
| | SWOLLEN?″: ″NO″ |
| 216 | }, |
| 217 | ″content″: ″″, |
| 218 | ″mergeLeft″: false |
| 219 | }, |
| 220 | { |
| 221 | ″id″: ″b14ba3f4-adcd- |
| | 40fe-a539- |
| | dbd5d4ce058f″, |
| 222 | ″link″: ″″, |
| 223 | ″choices″: { |
| 224 | ″HOW MANY JOINTS |
| | ARE INVOLVED?″: |
| | ″2-4″, |
| 225 | ″IS THE JOINT |
| | RED, HOT, |
| | SWOLLEN? ″: ″NO″ |
| 226 | }, |
| 227 | ″content″: ″″, |
| 228 | ″mergeLeft″: true |
| 229 | }, |
| 230 | { |
| 231 | ″id″: ″f5ae094c-f711- |
| | 48ba-8d85- |
| | 71f7b360ea75″, |
| 232 | ″link″: ″″, |
| 233 | ″choices″: { |
| 234 | ″HOW MANY JOINTS |
| | ARE INVOLVED?″: |
| | ″MORE THAN 4″, |
| 235 | ″IS THE JOINT |
| | RED, HOT, |
| | SWOLLEN?″: ″NO″ |
| 236 | }, |
| 237 | ″content″: ″″, |
| 238 | ″mergeLeft″: true |
| 239 | } |
| 240 | ], |
| 241 | ″content″: ″″, |
| 242 | ″tooltip″: ″″, |
| 243 | ″pageType″: ″swimlane″, |
| 244 | ″questions″: [ ] |
| 245 | }, |
| 246 | ″createdAt″: ″2022-07- |
| | 05T18:04:41.913Z″, |
| 247 | ″updatedAt″: ″2022-07-05T18:04:41.913Z″ |
| 248 | }, |
| 249 | { |
| 250 | ″id″: ″9997c4e1-afca-4c16-a794- |
| | 4b1e0192fd5a″, |
| 251 | ″isGuide″: false, |
| 252 | ″isRoot″: false, |
| 253 | ″persona″: { |
| 254 | ″id″: ″0c82dbce-1000-4c54-ad20- |
| | 18af4f383bdb″, |
| 255 | ″name″: ″Provider″ |
| 256 | }, |
| 257 | ″careCategory″: { |
| 258 | ″id″: ″4c6c897d-70de-4e4a-9a21- |
| | 15e99cf4b444″, |
| 259 | ″name″: ″Outpatient″ |
| 260 | }, |
| 261 | ″patientGroup″: { |
| 262 | ″id″: ″59b96b3d-d330-418d-8054- |
| | 773f5b3fb24c″, |
| 263 | ″name″: ″Adult″ |
| 264 | }, |
| 265 | ″name″: ″No signs of inflammation″, |
| 266 | ″content″: { |
| 267 | ″content″: ″\u003ch3\u003eJoint |
| | pain without evidence of |
| | inflammation (redness, |
| | warmth)\u003c/h3\u003e\n\u003cdiv |
| | \u003eOffer symptom relief with |
| | acetaminophen 500-1000 mg QID |
| | (unless liver disease), topicals |
| | (diclofenac |
| | gel).\u003c/div\u003e\n\u003cdiv\ |
| | u003eLower extremity pain may |
| | improve with weight |
| | loss.\u003c/div\u003e\n\u003cdiv\ |
| | u003eRestricted AROM/PROM |
| | suggests intraarticular |
| | pathology.\u003c/div\u003e\n\u003 |
| | cdiv\u003e\n\u003 |
| | cdiv\u003e\n\u003cdiv\u003e\n\u00 |
| | 3cdiv\u003e\u003c |
| | strong\u003eMechanical |
| | disease\u003c/strong\u003e |
| | (OA)\u003c/div\u003e\n\u003c/div\ |
| | u003e\n\u003cdiv\u003e\n\u003cul\ |
| | u003e\n\u003cli\u003ePainful, |
| | limited |
| | ROM\u0026nbsp;\u003c/li\u003e\n\u |
| | 003cli\u003ecrepitus\u0026nbsp;\u |
| | 003c/li\u003e\n\u003cli\u003eeffu |
| | sions\u0026nbsp;\u003c/li\u003e\n |
| | \u003cli\u003eHeberden and |
| | Bouchard Nodes (DIP, |
| | PIP)\u0026nbsp;\u003c/li\u003e\n\ |
| | u003c/ul\u003e\n\u003c/div\u003e\ |
| | n\u003c/div\u003e\n\u003cdiv\u003 |
| | e\n\u003cdiv\u003e\n\u003cul\u003 |
| | e\n\u003cli\u003edeformities\u002 |
| | 6nbsp;\u003c/li\u003e\n\u003c/ul\ |
| | u003e\n\u003cdiv\u003eCDC \u003ca |
| | class=\″external-link\″ |
| | href=\″https://www.cdc.gov/arthri |
| | tis/pain/index.htm\″ |
| | target=\″_blank\″rel=\″noopener\″ |
| | \u003erecommendations on |
| | arthritis\u003c/a\u003e\u003c/div |
| | \u003e\n\u003c/div\u003e\n\u003c/ |
| | div\u003e\n\u003c/div\u003e\n\u00 |
| | 3 ch3\u003eOrders to |
| | consider\u003c/h3\u003e\n\u003cdi |
| | v\u003eIf only a few joints are |
| | involved, consider Xray of |
| | affected |
| | joint.\u003c/div\u003e\n\u003cdiv |
| | \u003e\u003caction |
| | id=\″2380304364916750\″ |
| | class=\″actionNonEditable\″ |
| | title=\″Choosing Wisely\″ |
| | type=\″choosingwisely\″\u003e\u00 |
| | 3cactioncontent |
| | class=\″actionNonEditable\″ |
| | type=\″choosingwisely\″\u003eChoo |
| | sing |
| | Wisely\u003c/actioncontent\u003e\ |
| | u003c/action\u003 eIf foot or |
| | ankle films are ordered for |
| | arthritis evaluation, ensure they |
| | are weight bearing \u003ca |
| | class=\″external-link\″ |
| | href=\″https://www.choosingwisely |
| | .org/clinician-lists/american- |
| | orthopaedic-foot-ankle-society-x- |
| | rayevaluation-of-foot-and-ankle- |
| | without-standing/\″ |
| | target=\″_blank\″ |
| | rel=\″noopener\″\u003eChoosing |
| | Wisely\u003c/a\u003e\u0026nbsp;\u |
| | 003c/div\u003e\n\u003cdiv\u003e\u |
| | 003c/div\u003e\n\u003cdiv\u003e\u |
| | 003caction |
| | id=\″5130143634902115\″ |
| | class=\″actionNonEditable\″ |
| | title=\″Choosing Wisely\″ |
| | type=\″choosingwisely\″\u003e\u00 |
| | 3cactioncontent |
| | class=\″actionNonEditable\″ |
| | type=\″choosingwisely\″\u003e\u00 |
| | 3c/actioncontent\u003e\u003c/acti |
| | on\u003eDon\u0026rsquo;t test for |
| | Lyme disease as a cause of |
| | musculoskeletal symptoms without |
| | an exposure history and |
| | appropriate exam |
| | findings.\n\u003cdiv\u003e The |
| | musculoskeletal manifestations of |
| | Lyme disease sts/american- |
| | college-rheumatology-lyme- |
| | disease-testing-in-children- |
| | without-history/\″ |
| | target=\″_blank\″ |
| | rel=\″noopener\″\u003eChoosing |
| | Wisely\u003c/a\u003e\u003c/div\u0 |
| | 03e\n\u003cdiv\u003e\n\u003cdiv\u |
| | 003e\n\u003cdiv\u003e\n\u003cdiv\ |
| | u003e\u003c/div\u003e\n\u003c/div |
| | \u003e\n\u003cdiv\u003e\n\u003ch3 |
| | \u003e\u003cspan\u003e\u003cstron |
| | g\u003eReferral Scheduling |
| | Recommendations\u003c/strong\u003 |
| | e\u003c/span\u003e\u0026nbsp;\u00 |
| | 3c/h3\u003e\n\u003c/div\u003e\n\u |
| | 003cdiv\u003e\n\u003cdiv\u003e\n\ |
| | u003ch4\u003e\u003cspan\u003e\u00 |
| | 3cstrong\u003eRoutine\u003c/stron |
| | g\u003e\u003c/span\u003e (2-8 |
| | weeks):\u0026nbsp;\u003c/h4\u003e |
| | \n\u003c/div\u003e\n\u003cdiv\u00 |
| | 3e\n\u003cul\u003e\n\u003cliclass |
| | =\″li- |
| | arrow\″\u003e\u003cspan\u003e\u00 |
| | 3cstrong\u003ePT |
| | referral\u003c/strong\u003e\u003c |
| | /span\u003e for mild non |
| | rheumatic joint |
| | disease\u0026nbsp;\u003c/li\u003e |
| | \n\u003cliclass=\″li- |
| | arrow\″\u003e\u003cspan\u003e\u00 |
| | 3cstrong\u003eOrthopedics |
| | referral\u003c/strong\u003e\u003c |
| | /span\u003e for severe or |
| | refractory joint |
| | disease\u0026nbsp;\u003c/li\u003e |
| | \n\u003c/ul\u003e\n\u003cdiv\u003 |
| | eWould you like to see more about |
| | a specific |
| | joint?\u003c/div\u003e\n\u003c/di |
| | v\u003e\n\u003c/div\u003e\n\u003c |
| | /div\u003e\n\u003c/div\u003e\n\u0 |
| | 03c/div\u003e″, |
| 268 | ″tooltip″: ″″, |
| 269 | ″pageType″: ″multiple_questions″, |
| 270 | ″questions″: [ |
| 271 | { |
| 272 | ″id″: |
| | ″6297709255783711″, |
| 273 | ″type″: ″exclusive″, |
| 274 | ″options″: [ |
| 275 | { |
| 276 | ″id″: |
| | ″40110248461 |
| | 81732″, |
| 277 | ″link″: |
| | ″#Knee Pain |
| | Referral |
| | Guide″, |
| 278 | ″text″: |
| | ″Knee″, |
| 279 | ″summary |
| | text″: ″″ |
| 280 | }, |
| 281 | { |
| 282 | ″id″: |
| | ″49324041157 |
| | 05893″, |
| 283 | ″link″: |
| | ″#Hip Pain |
| | Referral |
| | Guide″, |
| 284 | ″text″: |
| | ″Hip″ |
| 285 | }, |
| 286 | { |
| 287 | ″id″: |
| | ″63162458032 |
| | 57777″, |
| 288 | ″link″: |
| | ″#Trochanter |
| | ic Bursitis |
| | Referral |
| | Guide″, |
| 289 | ″text″: |
| | ″Lateral |
| | hips″, |
| 290 | ″tooltip″: |
| | ″\u003cdiv\u |
| | 003eIf pain |
| | is isolated |
| | to lateral |
| | hip and |
| | tender to |
| | palpation, |
| | consider |
| | trochanteric |
| | bursitis\u00 |
| | 3c/div\u003e |
| | ″ |
| 291 | } |
| 292 | ], |
| 293 | ″question″: ″″, |
| 294 | ″exclusive_key″: ″WHICH |
| | JOINT ″ |
| 295 | } |
| 296 | ] |
| 297 | }, |
| 298 | ″createdAt″: ″2022-07- |
| | 05T18:04:41.889Z″, |
| 299 | ″updatedAt″: ″2022-07-05T18:29:09.018Z″ |
| 300 | } |
| 301 | ] |
| 302 | } |
| 303 | } |

Lines 1-90 of the internal representation provide information about the analytic method as a whole. Line 3 contains a unique identifier that can be used to distinguish the analytic method from other analytic methods. Line 4 shows the current stage of the analytic method within an analytic method production cycle. Line 5 shows the name specified for the analytic method. Lines 6-17 show tags that can be used to search for or otherwise locate this analytic method in situations where it may be needed. Lines 19-40 identify subject matter categories to which the analytic method belongs, which can be used to find the analytic method in situations in which it might be useful. Lines 41-62 identify people who have participated in creation, revision, proofreading, maintenance, etc. of the analytic method. Lines 63-66 identify a governance model for the analytic method. Lines 67-90 identify a variety of metadata for the analytic method, including creation and updating timestamps shown in lines 89-90.

Lines 91-301 specify the pages of the analytic method. In particular, lines 92-160 specify the Chronic Joint Pain page corresponding to node 2080; lines 161-248 specify the Jt pain swimlane page corresponding to node 2081; and lines 249-300 specify the No sign of inflammation page corresponding to node 2082.

Line 93 contains the unique identifier for the Chronic Joint Pain page. Line 94 indicates that this page is an active part of an analytic method, as opposed to a “guide” page constituting a plain document. Line 95 specifies that this page's node is the root of the graph, where the analytic method beings. Lines 96-99 identify the consumer type for which the analytic method is intended; lines 100-103 identify a patient care mode for which the analytic method; and lines 104-107 identify the patient type for which the analytic method is intended. Line 108 specifies the name of the page, which is shown in the display of the page and the page's graph node, and is used by other pages of the analytic method to refer to the page. Lines 109-110 specify the content of this page, shown at reference number 614 in FIG. 6, in an encoded form. Inside, tag delimiter symbols “<” and “>” are encoded using their ASCII values “/u003c” and “/u003e,” respectively. It can be seen that an action tag (“<action>”) specifies the supplemental information icon 611, as well as the following text to be displayed when the user hovers over it: “including septic, viral (COVID, Parvo), lyme, RMSF. Acute onset, fever, rash, arthritis, must r/o infectious diseases.” Line 111 specifies the tooltip text that is to be displayed if the user hovers anywhere over the page. Line 112 specifies that multiple question controls occur on the page. Line 113 specifies that this page routes to the Jt pain swimlane page after its questions are answered.

Line 114-156 specify the two questions in the page and their possible answers. For the first question, Line 136 specifies its text as “How many joints are involved?”. Lines 119-123 specify the first possible answer (“option”) for the first question as “1”; lines 124-129 specify as the second possible answer to the first question as “2-4”; and lines 130-134 specify the third possible answer to the first question as “more than 4.” Lines 138-155 similarly define the second question “Is the joint red, hot, swollen?” and its two possible answers, “Yes” and “No.”

For the Jt pain swimlane page defined in lines 161-248, lines 161-177 specify metadata for this page similar to the metadata discussed above in connection with the Chronic Joint Pain page. Also, line 243 identifies the type of the page as “swimlane,” i.e., one that performs differential routing based upon different combinations of answers to questions. Lines 180-189 relate to the first column 1355 of the swimlane; lines 183-185 indicate that this first column is for the combination of the answer “More than 4” to the question “How many joints are involved?” with the answer “Yes” to the question “Is the joint red, hot, swollen?”. Line 182 indicates that the swimlane does not route to a separate page corresponding to the empty next page field 1504 left by the creator in FIG. 15. Line 187 includes an encoded version of the content 1503 entered by the creator in FIG. 15. Line 188 shows that the first column has not been merged with any column to the left-indeed, there is no column to the left of the first column.

Lines 190-199 relate to the second column 1356 of the swimlane, which lines 193-195 relate to a selection of the answer “2-4” to the question “How many joints are involved?” with the answer “Yes” to the question “Is the joint red, hot, swollen?” Like the first column, line 192 specifies that the second column does not route to a separate page of the analytic method. Line 197 contains the encoded content to be displayed when the choices for the second column are selected.

Lines 200-209 relate to the third column 1357 of the swimlane, corresponding to the combination of the answer “1” to the question “How many joints are involved?” and the answer “Yes” to the question “Is the joint red, hot, swollen?.” Line 202 specifies that the third column does not route to a different page, and line 207 contains the content displayed when the combination of answers to which this column corresponds are given.

Lines 210-219 relate to the fourth column 1358 of the swimlane, corresponding to the combination of the answer “1” to the question “How many joints are involved” and the answer “No” to the question “Is the joint red, hot, swollen?.” Line 217 specifies that no content is displayed when the combination of answers to which this column corresponds is given, but line 212 specifies that, when this combination of answers is given, the facility should route to the “No signs of inflammation” page.

Lines 220-229 relate to the fifth column 1359 of the swimlane, corresponding to the combination of the answer “2-4” to the question “How many joints are involved?” with the answer “No” to the question “Is the joint red, hot, swollen?” Line 228 specifies that the fifth column has been merged with the column to its left, the fourth column. Therefore, when the fifth column's combination of answers is given, the facility routes to the “No signs of inflammation” page in accordance with line 211 mentioned above.

Lines 230-239 relate to the sixth column of the swimlane 1360, which corresponds to a combination of the answer “more than 4” to the question “How many joints are involved?” with the answer “No” to the question “Is the joint red, hot, swollen?”. Like the fifth column, line 238 marks the sixth column as being merged with the column to the left; thus, like the fourth and fifth columns, when the combination of answers to which the sixth column corresponds, the facility routes to the “No signs of inflammation” page.

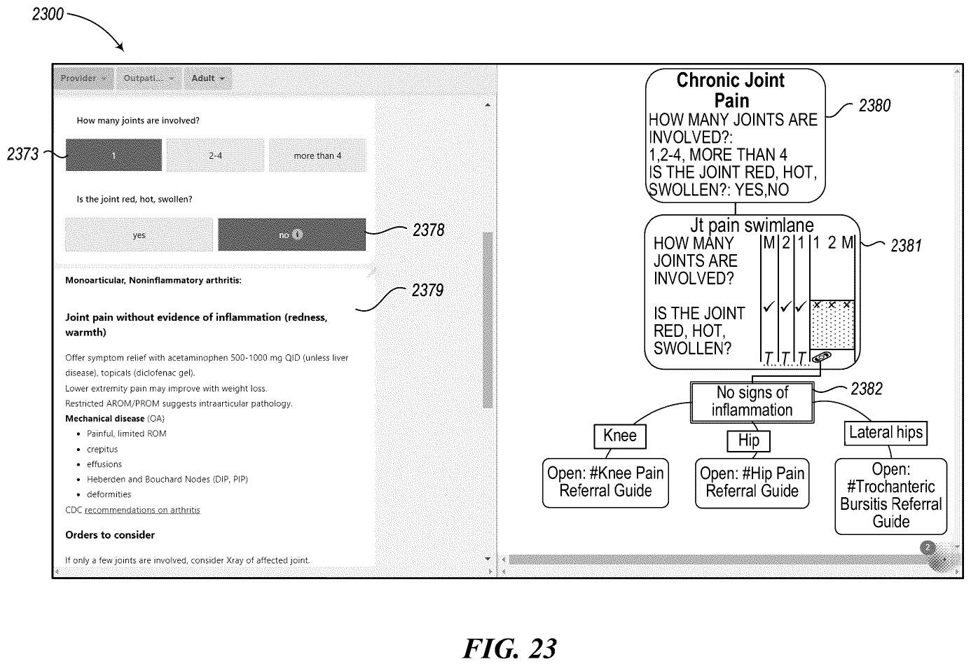

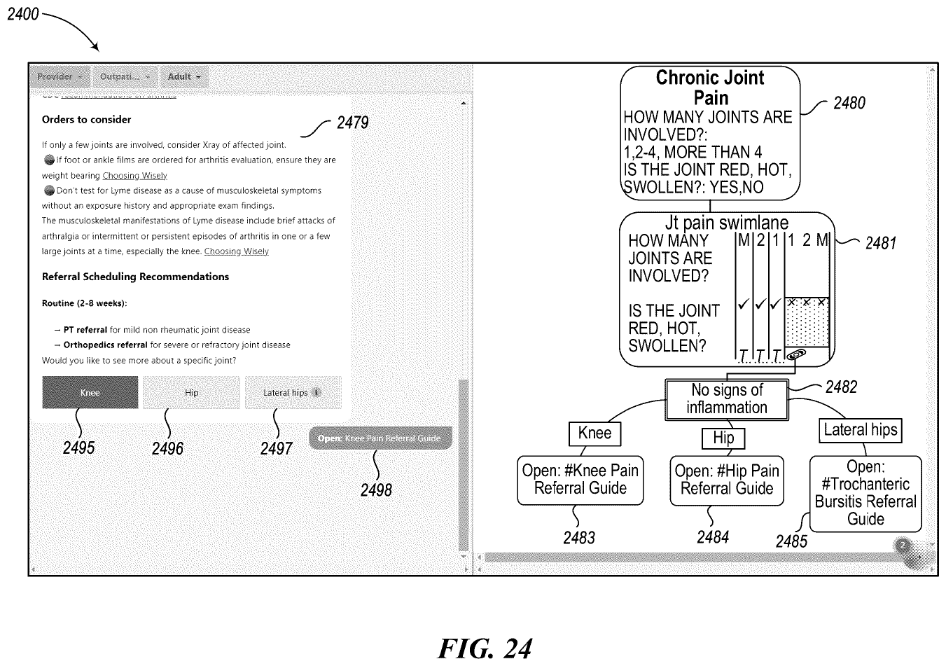

Lines 249-300 relate to the “No sign of inflammation” page—as indicated in line 265. Lines 250-264 contain metadata for this page. Line 267 contains the content of the page, shown as content 2379 and 2479 in FIGS. 23 and 24. Line 269 specifies that this page contains a question, which line 273 specifies has mutually exclusive possible answers—in other words, only one can be selected at a time. Lines 275-280 relate to the first answer to the question, “Knee” as specified in line 278. Line 277 specifies that, if this first answer is selected, the facility routes to the “#Knee Pain Referral Guide” document. Lines 281-285 relate to the second answer, “Hip” as specified in line 284. Line 283 specifies that, when the second answer is selected, the facility routes to the “#Hip Pain Referral Guide” document. Lines 286-291 relate to the third answer, specified as “Lateral hips” in line 288. Line 287 specifies that, when this answer is selected, the facility routes to the “#Trochanteric Bursitis Referral Guide” document. Also, line 290 specifies that this button has a supplemental information icon that, when hovered over, displays the text “If pain is isolated to lateral hip and tender to palpation, consider trochanteric bursitis.” While the question corresponding to these answers could have been specified in line 293, instead in this case it was specified near the end of the content for the page in line 267 as “Would you like to see more about a specific joint?”.

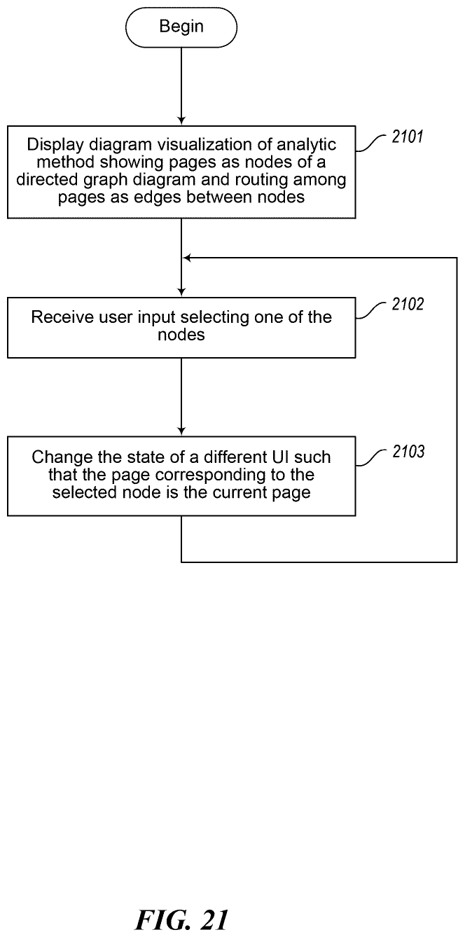

FIG. 21 is a flow diagram showing a process performed by the facility in some embodiments in order to permit navigation of the analytic method via the graph diagram. In act 2101, the facility displays a directed graph diagram visualization of an analytic method, which shows pages of the analytic method as nodes of a graph diagram, and shows routing among the pages as edges between nodes. The graph diagram shown in FIG. 20 is an example of such a graph diagram visualization. In act 2102, the facility receives user input selecting one of the nodes of the graph diagram, such as by touching it or clicking on it. In act 2103, the facility changes the state of a user interface other than the graph diagram to make the page corresponding to the node selected in the graph diagram the current page in the other user interface. In various embodiments, this other user interface could be the creator interface or one of the consumer interfaces. After act 2103, the facility continues in act 2102 to receive user input selecting another node of the graph diagram.

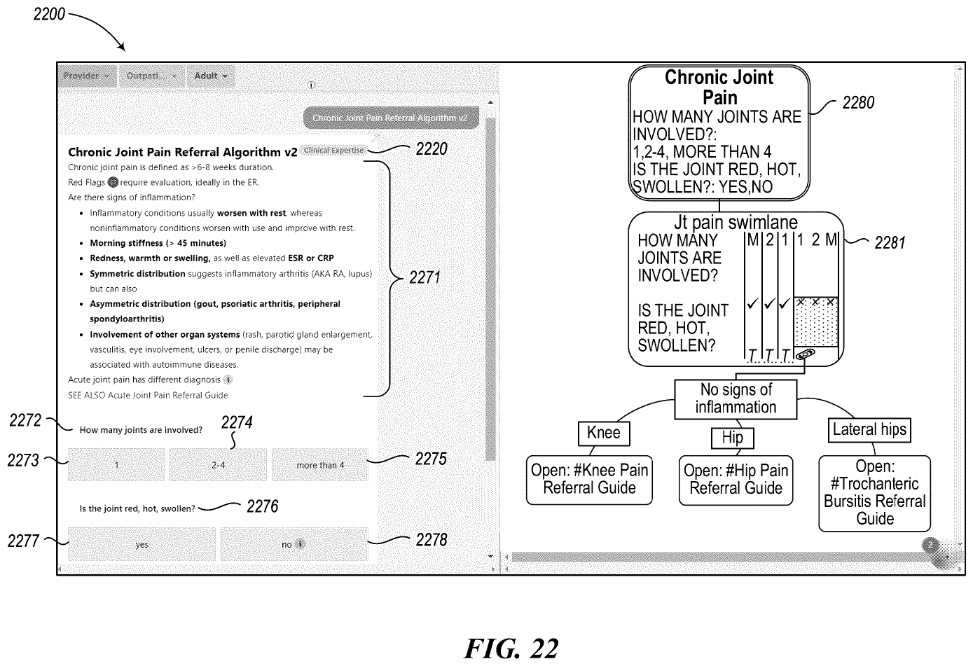

FIG. 22 is display diagram showing a sample display presented by the facility in some embodiments to begin navigating via the graph diagram visualization. Display 2200 shows the display after the user has clicked on node 2080 shown in the graph diagram that appears in FIG. 20. It can be seen that the corresponding node 2280, corresponding to the analytic method's starting page, has a green halo showing it to be the current node and showing the corresponding page of the analytic method to be the current page. Also, the facility has switched the display mode to show the consumer preview mode, which shows how pages of the analytic method will be displayed to the consumer as they are presently defined. It can be seen that the consumer view of the analytic method's first page 2270 is displayed, including content 2271, a first question 2272 and its possible answers 2273-2275, a second question 2276 and its possible answers 2277-2278. In this state, to navigate to the page corresponding to node 2281, the creator can either click on this node in the graph diagram, or answer the questions in the consumer mode of the creator user interface, such as by selecting possible answers 2273 and 2278. The effect of doing either is shown in FIG. 23 discussed below.

FIG. 23 is a display diagram showing a sample display presented by the facility to reflect navigation to the second page of the analytic method. It can be seen that the graph diagram node 2381 corresponding to the second page—the Jt pain swimlane page—has a green halo to indicate that this page is the current page. Also, the facility has displayed content 2379 for this page.

FIG. 24 is a display diagram showing sample contents of a display presented by the facility in some embodiments to navigate to the third page of the analytic method. It can be seen that the creator has scrolled down the contents of the second page 79 to see its question 94, and that question's possible answers 2495-2497. Because the user selected possible answer 2495, the facility indicates that the external “KneePainReferralGuide” document is to be opened. In some embodiments (not shown), the facility may add a halo to node 2483 to reflect this routing.

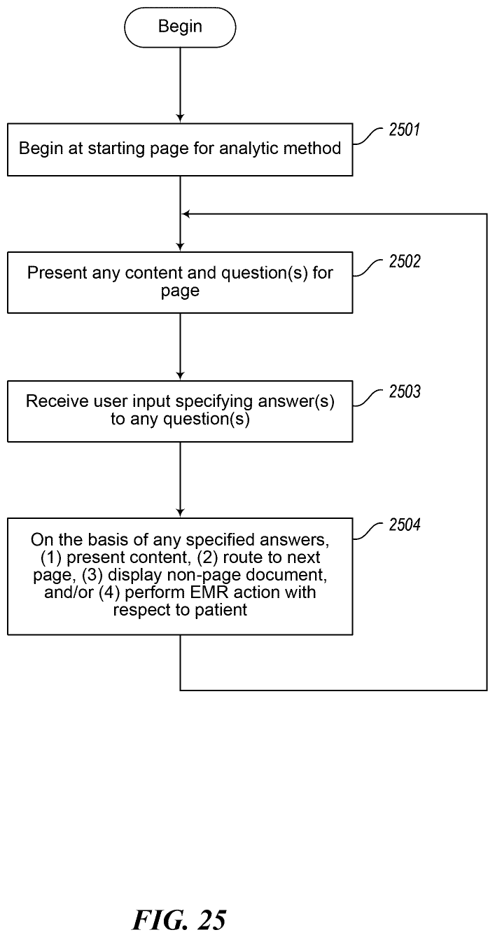

FIG. 25 is a flow diagram showing a process performed by the facility in some embodiments to apply an analytic method on behalf of a consumer user, such as one using the facility with respect to a particular patient. In act 2501, the facility begins at the starting page for the analytic method. In act 2502, the facility presents the content for the present page and any questions it contains. In some embodiments, the facility performs act 2502 by applying an interpreter to the internal representation of the analytic method. As discussed above, in various embodiments, the facility performs this presentation using a variety of output modalities. In act 2503, the facility receives user input specifying one or more answers to any questions presented in act 2502. As discussed above, in various embodiments, the facility receives this input using a variety of input modalities. In act 2504, on the basis of any answers specified in act 2503, the facility routes among multiple targets in accordance with the analytic method, such as presenting content specified by the creator for the specified answers, routing to a different page of the analytic method based on the specified answers, displaying a document external to the facility specified by the creator for the specified answers, and/or performing EMR action with respect to the patient specified by the creator for the specified answers. Such routing may also be unconditional, to a single target. After act 2504, the facility continues in act 2502 to present the next page and any content or questions it contains.

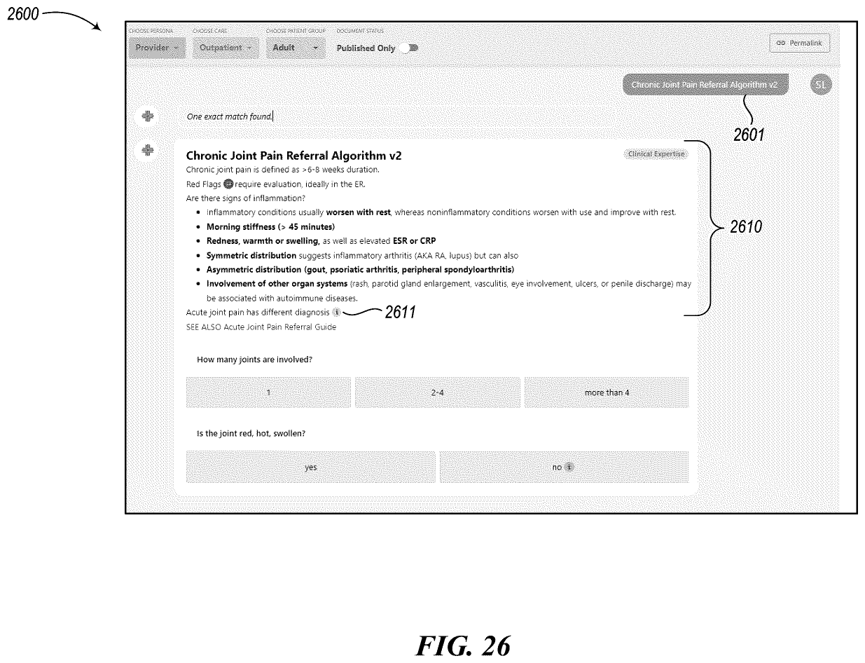

FIG. 26 is a display diagram showing a display presented by the facility in some embodiments to begin application of an analytic method. The display 2600 shows the page first of the sample analytic method whose definition is described above in connection with FIGS. 3-20, and whose internal representation is shown in Table 1. Its name 2601 is shown in the display, as is its content 2610. It can be seen that the content includes an information icon 2611 that the consumer can select in order to expand it, such as by clicking on it, touching it, or hovering over it. In some cases, similar information icons may be automatically expanded either every time they are displayed or in response to a certain state such as an answer to an earlier question or a property of the consumer or the patient.

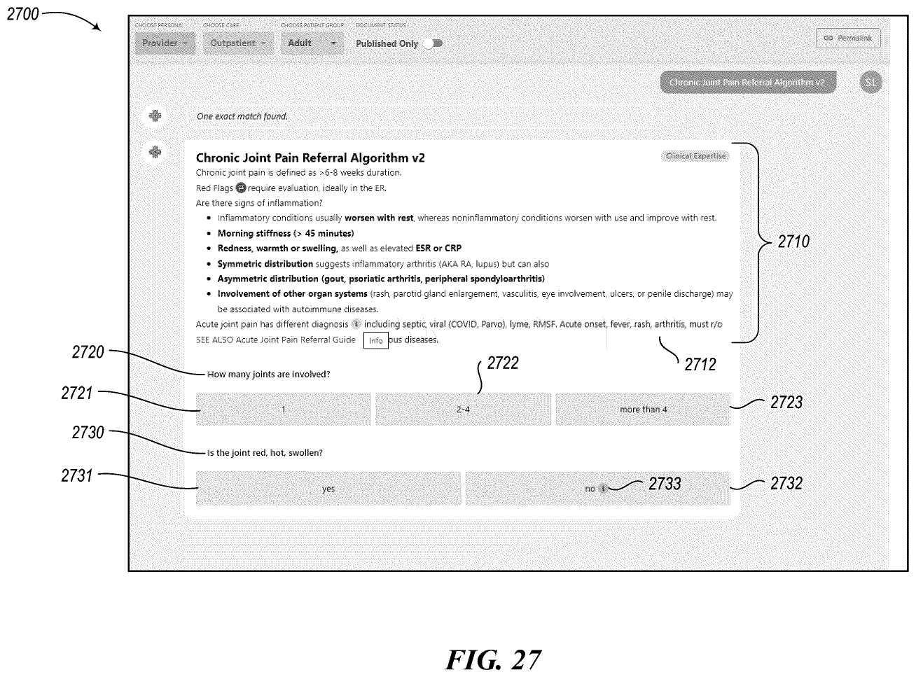

FIG. 27 is a display diagram showing a sample display presented by the facility in some embodiments to expand an information icon. In display 2700, it can be seen that the information icon 2611 has been expanded to show additional informational content 2712 relating to the portion of the content identified by the location of the information icon. The display also includes question 2720 and its possible answers 2721-2723, as well as question 2730 and its possible answers 2731 and 2732. It can be seen that possible answer 2733 has its own information icon, which can be similarly expanded to provide informational content about this possible answer.

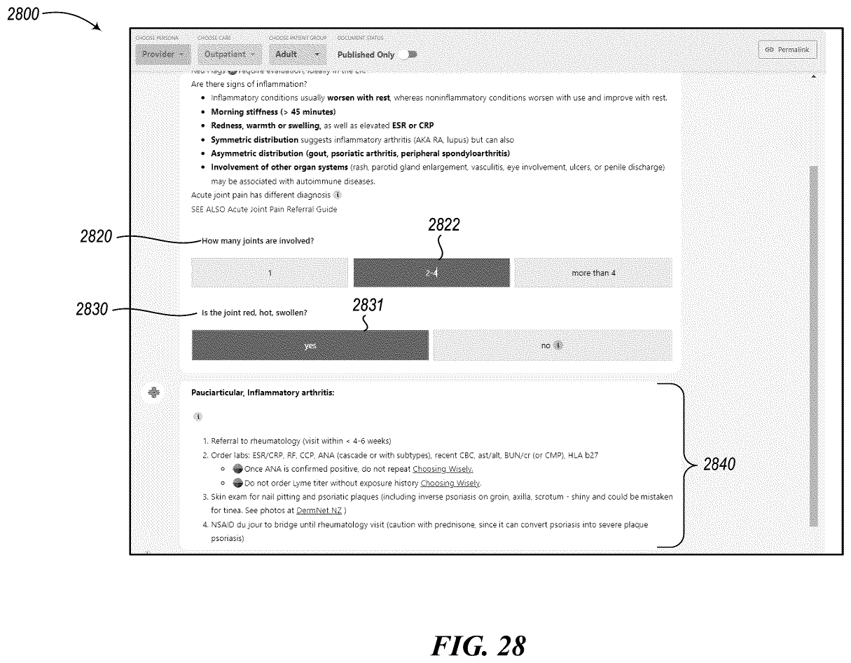

FIG. 28 is a display showing a sample display presented by the facility in some embodiments to display a second page of the sample analytic method in response to the consumer's answering of the questions of the first page. In particular, it can be seen in display 2800 that the consumer has selected answer 2822 to question 2820, and answer 2831 to question 2830. In response, the facility has displayed the content 2840 that the creator specified in FIG. 15 for the third column of swimlane 1350.

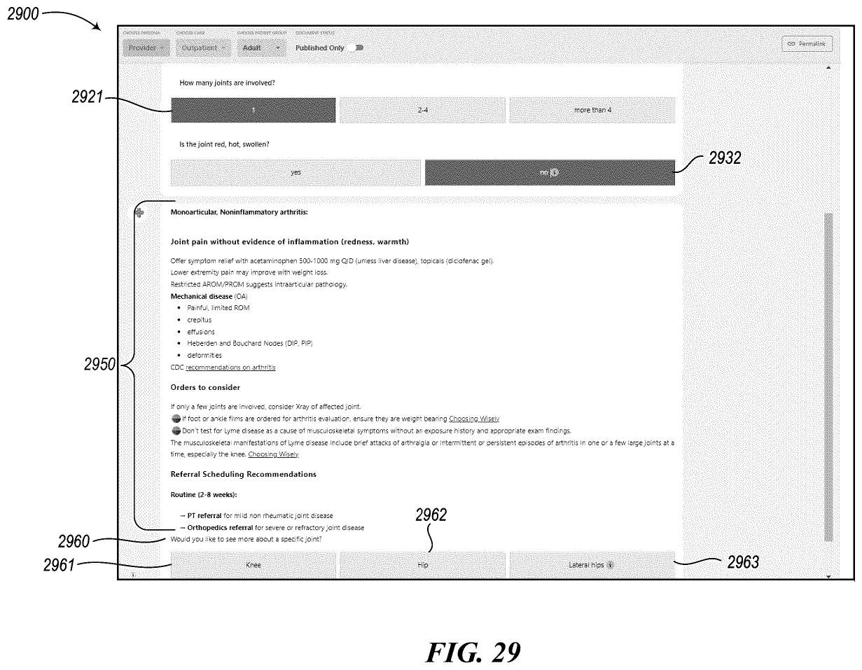

FIG. 29 is a display diagram showing a sample display presented by the facility in some embodiments to make visible the remainder of the second page of the sample analytic method. In particular, after scrolling down, display 2900 shows all of contents 2950 specified for the second page, as well as question 2960 and its possible answers 2961-2963, among which the consumer can select.

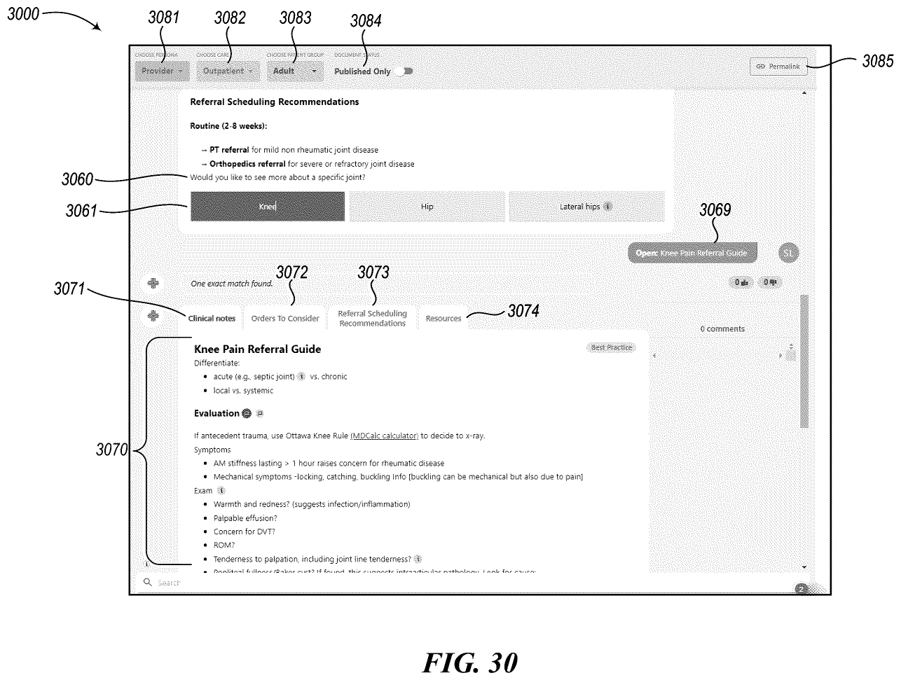

FIG. 30 is a display diagram showing a sample display presented by the facility in some embodiments to show the routing flow taken by the facility in the sample analytic method based upon the consumer's interactions in the third page. It can be seen that, in response to the consumer's selection of answer 3061 to question 3060, the facility has, in accordance with the creator's definition of that possible answer 2021 shown in FIG. 20, displayed the name 3069 of the external document specified by the creator for this possible answer, as well as the content 3070 of this external document. As shown, the external document is displayed in a tabbed interface, in which tabs 3071-3074 permit the consumer to select among different sections of the external document. In various embodiments, this display can be used by various other users, such as coauthors or non-author experts seeking to review the method.

In various embodiments, the facility supports various kinds of questions, and various approaches to routing in response to their answers. For example, in some embodiments, the facility prompts the consumer to enter a number, such as patient body mass index, and routes based on which of two or more numerical ranges the number falls within. In some embodiments, the facility prompts the consumer to provide a medical imaging artifact captured from the patient, and routes based on an automatic interpretation of the medical image artifact. In some embodiments, the facility prompts the consumer to provide a photograph or sketch of part of the patient's body, and routes based on an automatic analysis of this image. In some embodiments, the facility displays a diagram of a human body, prompts the consumer to touch or mark an affected area of the body on the diagram, and routes based upon the body area identified by the consumer. In some embodiments, the facility prompts the consumer to provide audio containing speech by the patient, and routes based on an automatic analysis of this audio.

In various embodiments, the facility supports a variety of types of routing targets. For example, in some embodiments, the facility supports routing to programs separate from the facility—such as those for displaying or interpreting medical imaging artifacts, those for making a live connection audio, video, and/or text connection with a person such as a specialist in a certain area of medicine, those for automatically generating text satisfying a text generation specification, etc.

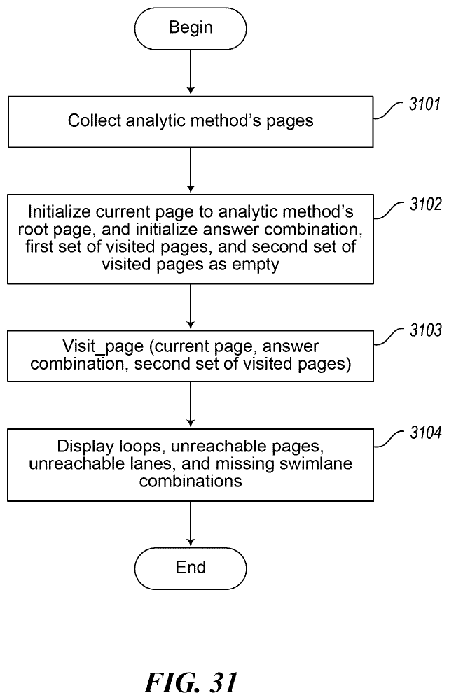

FIG. 31 is a flow diagram showing a process performed by the facility in some embodiments to perform a diagnostic assessment of an analytic method. In act 3101, the facility collects the pages of the analytic method. In act 3102, the facility initializes a current page of the analytic method to the analytic method's root page. The facility further initializes to empty both an answer combination and two sets of visited pages. In act 3103, the facility calls a visit_page function, passing the current page, the answer combination, and the second set of visited pages—as initialized in act 3102—as arguments. In act 3104, after the call to the visit_page function returns, the facility displays the results of the diagnostic assessment: any loops among the connections between pages in the analytic method, any pages of the analytic method that are unreachable from the root page, any lanes among the swimlane controls in the analytic method that correspond to impossible combinations of answers, and any combinations of answers that are missing from any of the swimlane controls in the analytic method. The facility does this based on the analytical results accumulated by recursive calls to the visit_page function. After act 3104, this process concludes.

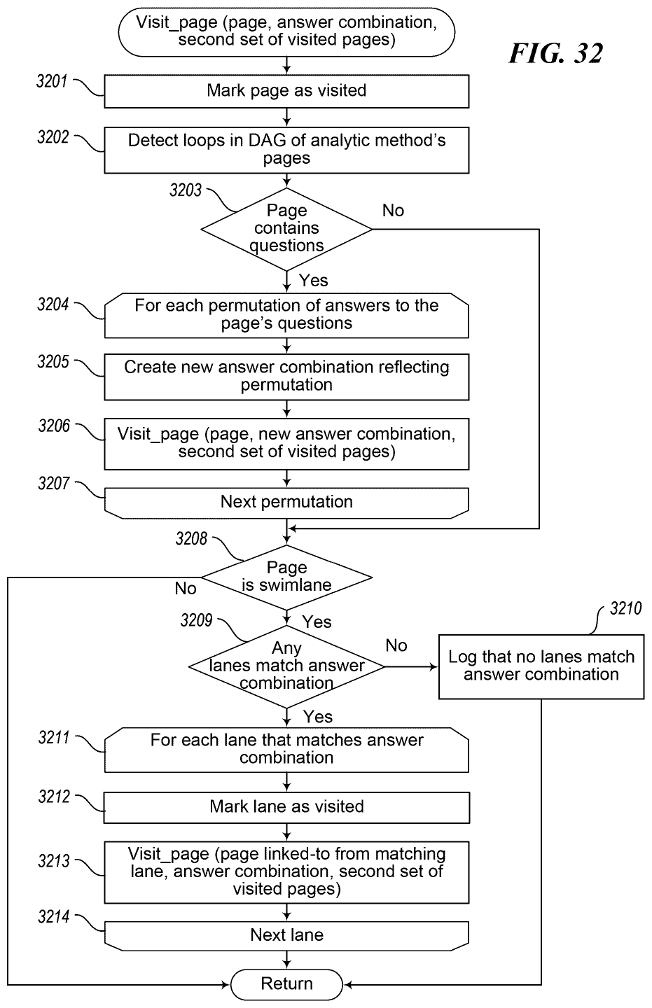

FIG. 32 is a flow diagram showing a process performed by the facility in some embodiments to handle a call to the visit_page function. The visit_page function receives arguments identifying a page that is being visited, an answer combination that is being tested, and a set of visited pages used to check for loops (the “second set of visited pages”). In act 3201, the facility marks the page identified by the page argument as visited, to exclude it from any list of unreachable pages in the analytic method. In act 3202, the facility detects any loops in the directed acyclic graph of the analytic method's pages, as connected by connections between pages. In act 3203, if the page contains any questions, then the facility continues in act 3204, else the facility continues in act 3208.

In acts 3204-3207, the facility loops through each permutation of answers to the questions contained by the page. In act 3205, the facility creates a new answer combination reflecting the current permutation in this loop iteration. In act 3206, the facility recursively calls the visit_page function, passing it the identity of the current page, as well as the new answer combination created in act 3205 in this iteration of the loop, and the second set of visited pages. In act 3207, if other permutations of answers remain to be processed, then the facility continues in act 3204 to process the next permutation of answers, else the facility continues in act 3208.

In act 3208, if the page is or contains a swimlane, then the facility continues in act 3209, else the visit_page function returns. In act 3209, if any lanes of the swimlane of the page match the answer combination that was the argument value in the call to the visit_page function, then the facility continues in act 3211, else the facility continues in act 3210. In act 3210, the facility logs that no lanes match the answer combination with which the visit_page function was called, identifying the combination as one for which a lane should be added to the swimlane in the analytic method. After act 3210, the call to the visit_page function returns.

In acts 3211-3214, the facility loops through each lane that matches the answer combination. In act 3212, the facility marks the lane as visited. In act 3213, the facility recursively calls the visit_page function with the argument values corresponding to the page that is linked-to from the matching swimlane that is the subject of the current iteration of the loop, and the answer combination. In act 3214, if additional matching lanes remain to be processed, then the facility continues in act 3211 to process the next matching swimlane, else the facility returns from this call to the visit_page function.

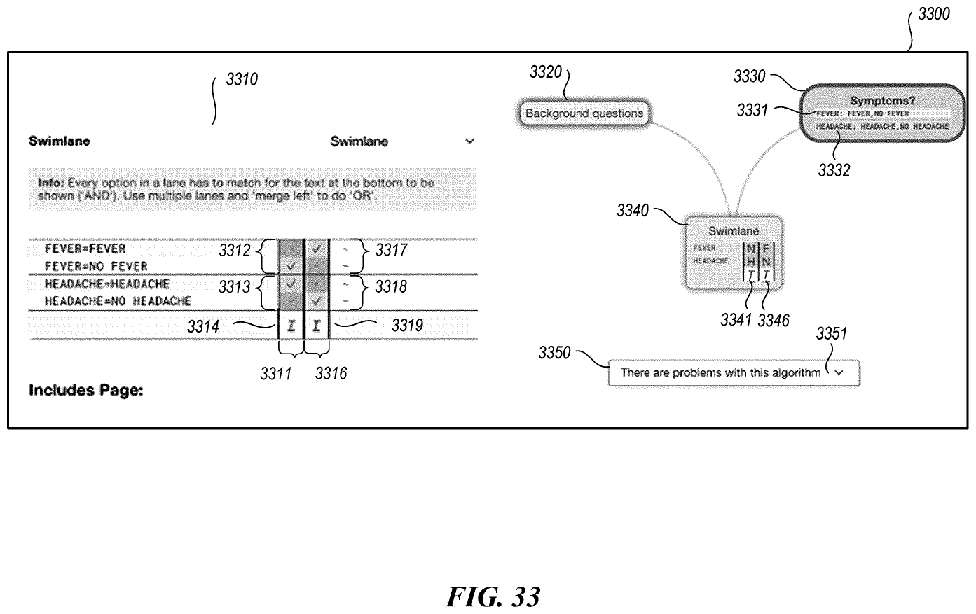

FIG. 33 is a display diagram showing a first display presented by the facility in some embodiments to show the results of the facility's diagnostic assessments for a sample analytic method. The display 3300 includes a graph of the pages making up the diagnostic method: a background questions page 3320, a symptoms questions page 3330, and a swimlane page 3340. The symptoms question page shows two questions 3231 and 3232 that it poses, as well as the possible answers to each of these questions. The swimlane page 3340 shows the two lanes that are presently defined for the swimlane control, lane 3341 and lane 3346. For example, lane 3341 performs routing within the analytic method for the combination of the answer no fever to the fever question and the answer headache to the headache question.

The display further includes an editing region 3310 for the swimlane control of the swimlane page. In particular, editing control shows lane 3311 corresponding to lane 3341 and lane 3316 corresponding to lane 3346. In lane 3311, it can be seen that this lane specifies the answer no fever 3312 to the fever question, and the answer headache 3313 to the headache question. This lane further includes a link 3314 to the page or other routing target that is routed-to by this lane of the swimlane, i.e., in iterations of the analytic method where the combination of answers of lane 3311 have been given.

The display also includes a compact notification 3350 that the facility's diagnostic assessment has identified errors with the analytic method in its present state. The user can activate control 3351 to expand the notification and receive details about the results of the diagnostic assessment.

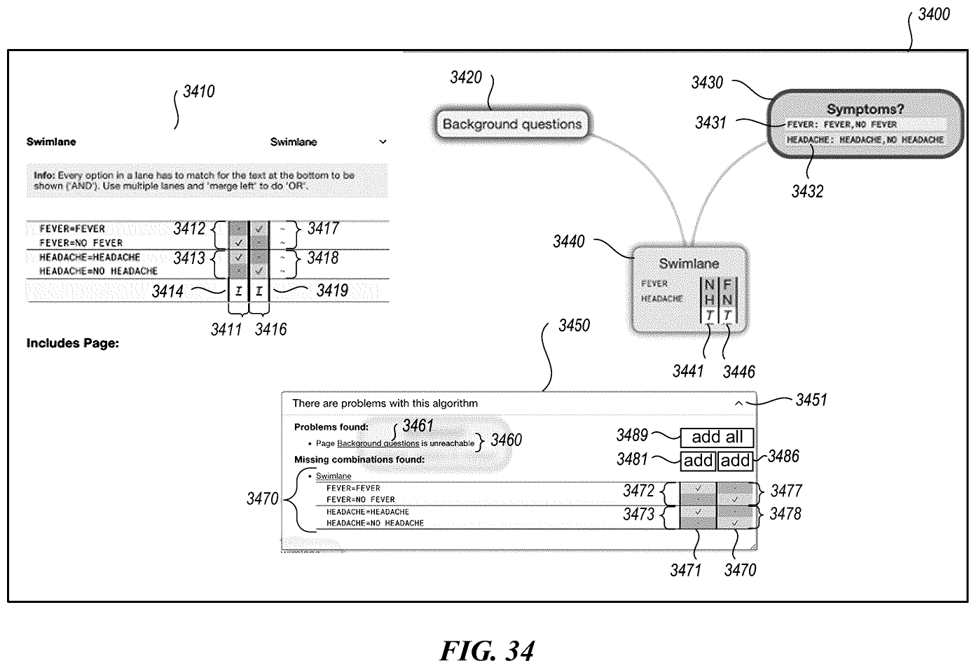

FIG. 34 is a display diagram showing a second display presented by the facility in some embodiments to show the results of the facility's diagnostic assessment for a sample analytic method. It can be seen that compact notification 3350 has been expanded to robust notification 3450 in response to the creator user activating control 3351. The expanded notification includes a control 3451 that the user can activate in order to collapse the notification. The notification further includes a section 3460 of errors related to connections between pages: any pages that are unreachable from the root page 3330—here, the background questions page 3320 identified by reference 3461—and any cycles or loops among the pages (none shown). In some embodiments, the reference 3461 to the background questions page is a link that the creator user can follow, such as to display the editing view of the background questions page, or other controls that the creator user can use to make the background questions page reachable either directly or indirectly from a root page of the analytic method.

The expanded notification further contains a section 3470 showing missing combinations of answers for which the facility can add lanes to the swimlane. In particular, the sections shows missing combinations of answers 3471 and 3476. The creator user can activate control 3481 to add a lane to the swimlane for missing combination 3471; activate control 3486 to add a lane for missing combination 3476; or activate control 3489 to add lanes to the swimlane for both missing combinations.

In some embodiments, the facility permits creators to specify one or more additional components of a swimlane. A first such additional component-a “supplemental content” additional swimlane component-available to creators in some embodiments is text or other content, or a reference to a page containing such content, that is always displayed to the consumer in addition to content displayed based on the matching lane of the swimlane. The presence in a swimlane of a supplemental content swimlane component does not affect the facility's checking for missing lanes in a swimlane.

A second such additional component—a “default destination” additional swimlane component—available to creators in some embodiments is a destination that the swimlane routes to in the event that no lane of the swimlane matches the combination of answers given by the consumer. The presence in a swimlane of a default destination swimlane component does affect facility's checking for missing lanes in a swimlane: once a default destination swimlane component is added to a swimlane, the swimlane can no longer have missing lanes, because the swimlane will have the effect of routing for any possible combination of answers.

In various embodiments, the facility uses various approaches in the creator user interface to enable creators to specify supplemental content and/or default destination swimlane components for a swimlane, including separate fields for specifying the destinations for these components, or additional columns for these components that specify their destinations. In various embodiments, the facility uses various approaches to identifying a column of the swimlane as an additional column representing a specify supplemental content or default destination components, such as adding additional rows to the swimlane controls for supplemental content and/or default destination, whose checking in a particular column specifies that that column has the corresponding significance.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system, comprising:
accessing an analytic method definition, the definition specifying a set of pages, one or more of the pages of the set that each include one or more questions, having two or more possible answers, one or more of the pages each including a swimlane control, each swimlane control comprising one or more lanes, each lane identifying a combination of possible answers to two or more of the questions included among the pages and specifying routing in an iteration of the analytic method in which that combination of answers has been given;
for each of the swimlane controls:
among the included questions any of whose answers have been included in the combination of answers identified by a lane of the swimlane, determining any combinations of possible answers not identified by any lane of the swimlane; and
causing to be displayed visual indications of at a least a portion of the combinations of possible answers determined for the swimlane control.

2. The method of claim 1, further comprising:
for each visual indication, causing to be displayed a control that can be activated to add to the corresponding swimlane control a lane identifying the indicated combination of possible answers.

3. The method of claim 1, further comprising:
causing to be displayed a control that can be activated to add to each corresponding swimlane control a lane identifying each of the indicated combination of possible answers.

4. The method of claim 1 wherein the analytic method definition further specifies directed connections each connecting a source page among the set of pages to a different destination page among the set of pages,
the method further comprising:
identifying a group of pages of the set among which connections establish a loop, such that, for each member of the group, a series of connections can be followed to leave from the member, then return to the member; and
causing to be displayed a visual indication identifying the pages of the group.

5. The method of claim 1 wherein the analytic method definition further specifies directed connections each connecting a source page among the set of pages to a different destination page among the set of pages,
and wherein the analytic method definition further designates a page among the set of pages as a root page at which each iteration of the analytic method begins,
the method further comprising:
identifying a page among the set of pages that cannot be reached by following connections from the root page; and
causing to be displayed a visual indication of the identified page.

6. The method of claim 1 wherein the analytic method definition further designates a page among the set of pages as a root page at which each iteration of the analytic method begins,
and wherein the determining is performed by a calling a function with respect to the root page, which recursively calls itself with respect to other pages of the set of pages.

7. The method of claim 6 wherein each call to the function provides values of a first parameter identifying a page among the set of pages with respect to which the function is to be performed, and a second parameter identifying a combination of possible answers to which the function is to be performed.

8. One or more instances of computer-readable media collectively containing a data structure relating to an analytic method definition, the definition specifying a set of pages, one or more of the pages of the set that each include one or more questions, having two or more possible answers, one or more of the pages each including a swimlane control, each swimlane control comprising one or more lanes, each lane identifying a combination of possible answers to two or more of the questions included among the pages and specifying routing in an iteration of the analytic method in which that combination of answers has been given, the data structure comprising:
one or more first entries, each first entry comprising:
information identifying one of the swimlane controls included among the pages of the set; and
information identifying a combination of possible answers that is not identified by any of the lanes of the swimlane control identified by the first entry, each answer of the identified combination corresponding to a question any of whose answers has been included in any lane of the identified swimlane control,
such that the contents of the data structure are usable to generate a notification of lanes missing from any of the swimlane controls included among the pages of the set.

9. The one or more instances of computer-readable media of claim 8 wherein the analytic method definition further specifies directed connections each connecting a source page among the set of pages to a different destination page among the set of pages,
the data structure further comprising:
a second entry identifying a group of pages of the set among which connections establish a loop, such that, for each member of the group, a series of connections can be followed to leave from the member, then return to the member.

10. The one or more instances of computer-readable media of claim 8 wherein the analytic method definition further specifies directed connections each connecting a source page among the set of pages to a different destination page among the set of pages,
and wherein the analytic method definition further designates a page among the set of pages as a root page at which each iteration of the analytic method begins,
the data structure further comprising:
a second entry identifying a page among the set of pages that cannot be reached by following connections from the root page.

11. One or more instances of computer-readable media collectively having contents configured to cause a computing system to perform a method, the method comprising:
accessing an analytic method definition, the definition specifying a set of pages, one or more of the pages of the set that each include one or more questions, having two or more possible answers, one or more of the pages each including a swimlane control, each swimlane control comprising one or more lanes, each lane identifying a combination of possible answers to two or more of the questions included among the pages and specifying routing in an iteration of the analytic method in which that combination of answers has been given; and
for each of the swimlane controls:
among the included questions any of whose answers have been included in the combination of answers identified by a lane of the swimlane, determining any combinations of possible answers not identified by any lane of the swimlane.

12. The one or more instances of computer-readable media of claim 11, the method further comprising:
causing to be displayed visual indications of at a least a portion of the combinations of possible answers determined for the swimlane control.

13. The one or more instances of computer-readable media of claim 11, the method further comprising:
for each visual indication, causing to be displayed a control that can be activated to add to the corresponding swimlane control a lane identifying the indicated combination of possible answers.

14. The one or more instances of computer-readable media of claim 11, the method further comprising:
causing to be displayed a control that can be activated to add to each corresponding swimlane control a lane identifying each of the indicated combination of possible answers.

15. The one or more instances of computer-readable media of claim 11 wherein the analytic method definition further specifies directed connections each connecting a source page among the set of pages to a different destination page among the set of pages,
the method further comprising:
identifying a group of pages of the set among which connections establish a loop, such that, for each member of the group, a series of connections can be followed to leave from the member, then return to the member; and
causing to be displayed a visual indication identifying the pages of the group.

16. The one or more instances of computer-readable media of claim 11 wherein the analytic method definition further specifies directed connections each connecting a source page among the set of pages to a different destination page among the set of pages,
and wherein the analytic method definition further designates a page among the set of pages as a root page at which each iteration of the analytic method begins,
the method further comprising:
identifying a page among the set of pages that cannot be reached by following connections from the root page; and
causing to be displayed a visual indication of the identified page.

17. The one or more instances of computer-readable media of claim 11 wherein the analytic method definition further designates a page among the set of pages as a root page at which each iteration of the analytic method begins,
and wherein the determining is performed by a calling a function with respect to the root page, which recursively calls itself with respect to other pages of the set of pages.

18. The one or more instances of computer-readable media of claim 17 wherein each call to the function provides values of a first parameter identifying a page among the set of pages with respect to which the function is to be performed, and a second parameter identifying a combination of possible answers to which the function is to be performed.
